(19) United States
(12) Patent Application Publication
(10) Pub. No.: US 2026/0033850 A1
(43) Pub. Date: Feb. 5, 2026

(54) INTRAVASCULAR LITHOTRIPSY CATHETER WITH OMNI-DIRECTIONAL SHOCK WAVE EMITTERS

(71) Applicant: Shockwave Medical, Inc., Santa Clara, CA (US)

(72) Inventors: Tommy NGUYEN, San Jose, CA (US); Khanh VO, Daly City, CA (US)

(73) Assignee: Shockwave Medical, Inc., Santa Clara, CA (US)

(21) Appl. No.: 18/791,224

(22) Filed: Jul. 31, 2024

(51) Int. Cl. A61B 17/22; A61B 17/00

(52) U.S. Cl. CPC A61B 17/22022; A61B 17/00234; A61B 2017/00154; A61B 2017/00238; A61B 2017/00292; A61B 2017/00477; A61B 2017/00831; A61B 2017/22025; A61B 2017/22028; A61B 2017/22064

(57) ABSTRACT

An exemplary catheter for generating shock waves includes a catheter body; an enclosure mounted to the catheter body; an emitter band positioned at least partially around the catheter body and within the enclosure; and at least three electrodes positioned adjacent to the emitter band and spaced apart from the emitter band by respective spark gaps, the at least three electrodes together with the emitter band forming electrode pairs of at least three shock wave emitters of the emitter band, wherein at least one of the at least three shock wave emitters can be driven separately from at least one other of the at least three shock wave emitters.

### FIELD

The present disclosure relates generally to the field of medical devices and methods, and more specifically to shock wave catheter devices for treating calcified lesions in body lumens, such as calcified lesions and occlusions in vasculature and kidney stones in the urinary system.

### BACKGROUND

A wide variety of catheters have been developed for treating calcified lesions, such as calcified lesions in vasculature associated with arterial disease. For example, treatment systems for percutaneous coronary angioplasty or peripheral angioplasty use angioplasty balloons to dilate a calcified lesion and restore normal blood flow in a vessel. In these types of procedures, a catheter carrying a balloon is advanced into the vasculature along a guide wire until the balloon is aligned with calcified plaques. The balloon is then pressurized (normally to greater than 10 atm), causing the balloon to expand in a vessel to push calcified plaques back into the vessel wall and dilate occluded regions of vasculature.

More recently, the technique and treatment of intravascular lithotripsy (IVL) has been developed, which is an interventional procedure to modify calcified plaque in diseased arteries. The mechanism of plaque modification is through use of a catheter having one or more acoustic shock wave generating sources that can generate acoustic shock waves that modify the calcified plaque. IVL devices vary in design with respect to the energy source used to generate the acoustic shock waves, with two exemplary energy sources being electrohydraulic generation and laser generation.

For electrohydraulic generation of acoustic shock waves, a conductive fluid (e.g., saline) may be contained within an enclosure that surrounds electrodes or can be flushed through a tube that surrounds the electrodes. The calcified plaque modification is achieved by creating acoustic shock waves within the catheter by an electrical discharge across the electrodes. This energy discharge enters the surrounding fluid faster than the speed of sound and therefore generates the acoustic shock wave. In addition, the energy rapidly heats the fluid thereby creating one or more rapidly expanding vapor bubbles. These shock waves propagate radially outward and modify calcified plaque within the blood vessels. For laser generation of acoustic shock waves, a laser pulse is transmitted into and absorbed by a fluid within the catheter. This absorption process rapidly heats and vaporizes the fluid, thereby generating the rapidly expanding vapor bubble, as well as the acoustic shock waves that propagate outward and modify the calcified plaque. The acoustic shock wave intensity is higher if a fluid is chosen that exhibits strong absorption at the laser wavelength that is employed. These examples of IVL devices are not intended to be a comprehensive list of potential energy sources to create IVL shock waves.

The IVL process may be considered different from standard atherectomy procedures in that it cracks calcium but does not liberate the cracked calcium from the tissue. Hence, generally speaking, IVL should not require aspiration nor embolic protection. Further, due to the compliance of a normal blood vessel and non-calcified plaque, the shock waves produced by IVL do not modify the normal vessel tissue or non-calcified plaque. Moreover, IVL does not carry the same degree of risk of perforation, dissection, or other damage to vasculature as atherectomy procedures or angioplasty procedures using cutting or scoring balloons.

More specifically, catheters to deliver IVL therapy have been developed that include pairs of electrodes for electrohydraulically generating shock waves inside an angioplasty balloon. Shock wave devices can be particularly effective for treating calcified plaque lesions because the acoustic pressure from the shock waves can crack and disrupt lesions near the angioplasty balloon without harming the surrounding tissue. In these devices, the catheter is advanced over a guidewire through a patient's vasculature until it is positioned proximal to and/or aligned with a calcified plaque lesion in a body lumen. The balloon is then inflated with conductive fluid (using a relatively low pressure of 2-4 atm) so that the balloon expands to contact the lesion but is not an inflation pressure that substantively displaces the lesion. Voltage pulses can then be applied across the electrodes of the electrode pairs to produce acoustic shock waves that propagate through the walls of the angioplasty balloon and into the lesions. Once the lesions have been cracked by the acoustic shock waves, the balloon can be expanded further to increase the cross-sectional area of the lumen and improve blood flow through the lumen. Alternative devices to deliver IVL therapy can be within a closed volume other than an angioplasty balloon, such as a cap, balloons of variable compliancy, or other enclosure.

Hard-to-crack calcifications often found in coronary arterial beds, such as those having eccentric and nodular morphologies, however, have been difficult to treat using the aforementioned devices. Existing shock wave devices typically include shock wave emitters spaced along the length (i.e., along the longitudinal axis) of the device's body and are used to treat buildup of calcified plaque along the length of the inner wall of a body lumen such as a blood vessel. Such devices may include two emitters positioned 180 degrees apart from one another around the circumference of the catheter body. Such emitter configurations may generate the greatest shock waves force at only two circumferential locations. To effectively treat dense eccentric or nodular calcifications with such emitter configurations, physicians may need to rotate the catheter within the body lumen to align the locations of the greatest shock wave force with the calcifications, which may be difficult.

### SUMMARY

Described herein are systems, devices, and methods for generating shock waves at a plurality of circumferential locations using at least three shock wave emitters spaced around the circumference of a catheter. Including more than two shock wave emitters spaced around the circumference of a catheter at a given longitudinal location enables closer spacing between the respective shock wave emitters (e.g., spaced at 120-degrees, 90-degrees, 72 degrees, 60 degrees, etc.) about the circumference of the catheter. By decreasing the circumferential spacing between the shock wave emitters, the treatment effectiveness of shock waves generated by the shock wave emitters can be enhanced via increased constructive interference relative to two 180-degree spaced emitters while preserving an even distribution of emitters about the circumference of the catheter.

Catheters described herein may include at least one shock wave emitter band that forms at least three shock wave emitters that are circumferentially and/or axially spaced from one another. Each shock wave emitter can be formed by a portion of the emitter band that is aligned with a respective electrode. The electrode is spaced apart from the band by a spark gap such that a suitable voltage pulse applied across the emitter band and the respective electrode causes a shock wave to form at the shock wave emitting location. Thus, shock waves can be generated at three or more different circumferential locations of the emitter band, enabling the device to more uniformly treat multiple locations around its circumference without requiring rotation of the catheter. At least two shock wave emitters of the emitter band may be connected to different power supply channels, enabling the two shock wave emitters to be driven independently of one another. Additionally, at least one shock wave emitter connected to a ground terminal may be connected in series with those two shock wave emitters such that pulsing either of the supply channels results in simultaneous generation of shock waves from at least two different circumferential locations. Accordingly, various catheters described herein may be configured to generate shock waves from different combinations of at least two circumferential locations depending on which supply channel is used.

According to an aspect, an exemplary catheter for generating shock waves comprises: a catheter body; an enclosure mounted to the catheter body; an emitter band positioned at least partially around the catheter body and within the enclosure; and at least three electrodes positioned adjacent to the emitter band and spaced apart from the emitter band by respective spark gaps, the at least three electrodes together with the emitter band forming electrode pairs of at least three shock wave emitters of the emitter band, wherein at least one of the at least three shock wave emitters can be driven separately from at least one other of the at least three shock wave emitters.

Optionally, a first shock wave emitter is electrically connected in series to a second shock wave emitter of the at least three shock wave emitters, a third shock wave emitter is electrically connected in series to the second shock wave emitter, and the first shock wave emitter and the third shock wave emitter are not electrically connected in series. Optionally, a fourth shock wave emitter of the at least three shock wave emitters is electrically connected in series to the second shock wave emitter. Optionally, a first electrode is connected to a first channel of a switching circuit by a first supply wire extending along the catheter body, and a second electrode is connected to a second channel of a switching circuit by a second supply wire extending along the catheter body.

Optionally, the emitter band comprises at least three apertures corresponding to the at least three electrodes. Optionally, the at least three apertures are respectively spaced apart 120-degrees from one another in a circumferential direction about the emitter band. Optionally, the emitter band comprises four apertures spaced apart 90 degrees from one another in a circumferential direction about the emitter band. Optionally, the four apertures correspond to four shock wave emitters. Optionally, at least two of the at least three shock wave emitters are spaced apart from one another in a circumferential direction about the emitter band by any of 60 degrees, 72 degrees, 90 degrees, or 120 degrees. Optionally, at least two of the at least three shock wave emitters are spaced apart from one another in a circumferential direction about the emitter band by a range of 60 degrees to less than 180 degrees.

Optionally, the exemplary catheter comprises a second emitter band that forms at least one electrode of at least one shock wave emitter of the second emitter band, wherein at least one of the at least three electrodes is electrically connected to an electrode of the at least one shock wave emitter of the second emitter band. Optionally, the second emitter band is positioned distally of the emitter band. Optionally, at least one of the at least one shock wave emitter of the second emitter band is configured to be driven independently of at least one other shock wave emitter of the second emitter band. Optionally, at least one of the at least three shock wave emitters is positioned distally of at least one other of the at least three shock wave emitters. Optionally, the enclosure is a balloon configured to be filled or inflated with a conductive fluid. Optionally, the at least three electrodes respectively comprise a conductive portion of at least three insulated wires.

Optionally, the emitter band is at least 3 millimeters long. Optionally, a first shock wave emitter is axially offset from a second shock wave emitter by 1 millimeter to 5 millimeters. Optionally, a first shock wave emitter is axially offset from a second shock wave emitter by 1 millimeter to 3 millimeters. Optionally, the emitter band comprises a proximal band portion, a distal band portion, and a connecting region between the proximal band portion and the distal band portion. Optionally, the connecting region comprises a bracket that connects the proximal band portion and the distal band portion, wherein the proximal band portion forms a cylindrical proximal band portion and the distal band portion forms a cylindrical distal band portion. Optionally, the emitter band comprises a plurality of openings positioned between a first shock wave emitter of the at least three shock wave emitters and a second shock wave emitter of the at least shock wave emitters. Optionally, the plurality of openings are positioned distally of the first shock wave emitter and proximally of the second shock wave emitter. Optionally, the plurality of openings are formed using a laser cutter. Optionally, the emitter band comprises a plurality of thinned regions positioned between a first shock wave emitter of the at least three shock wave emitters and a second shock wave emitter of the at least shock wave emitters Optionally, the plurality of thinned regions are positioned distally of the first shock wave emitter and proximally of the second shock wave emitter.

According to an aspect, an exemplary catheter comprises: a catheter body, an enclosure mounted to the catheter body; an emitter band positioned at least partially around the catheter body and within the enclosure; and a first shock wave emitter connected to a first channel of a power supply, the first shock wave emitter comprising at least one electrode pair, the at least one electrode pair comprising the emitter band; and a second shock wave emitter connected to a second channel of the power supply so that the second shock wave emitter can be driven independently of the first shock wave emitter, the second shock wave emitter comprising at least one electrode pair that comprises the emitter band.

Optionally, the catheter comprises a third shock wave emitter connected to a ground terminal, the third shock wave emitter comprising at least one electrode pair, the at least one electrode pair comprising the emitter band. Optionally, the emitter band comprises at least three apertures corresponding to the first shock wave emitter, the second shock wave emitter, and the third shock wave emitter. Optionally, the at least three apertures are respectively spaced apart 120-degrees from one another in a circumferential direction about the emitter band. Optionally, the emitter band comprises four apertures spaced apart 90 degrees from one another in a circumferential direction about the emitter band. Optionally, the four apertures correspond to four shock wave emitters.

Optionally, at least two of the first shock wave emitter, the second shock wave emitter, and the third shock wave emitter are spaced apart from one another in a circumferential direction about the emitter band by any of 60 degrees, 72 degrees, 90 degrees, or 120 degrees. Optionally, at least two of the four shock wave emitters are spaced apart from one another in a circumferential direction about the emitter band by any of 60 degrees, 72 degrees, 90 degrees, or 120 degrees. Optionally, at least two of the first shock wave emitter, the second shock wave emitter, and the third shock wave emitter are spaced apart from one another in a circumferential direction about the emitter band by a range of 60 degrees to less than 180 degrees. Optionally, at least two of the four shock wave emitters are spaced apart from one another in a circumferential direction about the emitter band by a range of 60 degrees to less than 180 degrees.

Optionally, the catheter comprises a second emitter band electrically connected to the emitter band. Optionally, the second emitter band forms at least one electrode of at least one shock wave emitter of the second emitter band, wherein at least one electrode of the first shock wave emitter, the second shock wave emitter, and the third shock wave emitter is electrically connected to an electrode of the at least one shock wave emitter of the second emitter band. Optionally, the second emitter band is positioned distally of the emitter band. Optionally, at least one of the at least one shock wave emitter of the second emitter band is configured to be driven independently of at least one other shock wave emitter of the second emitter band.

Optionally, at least one of the first shock wave emitter, the second shock wave emitter, and the third shock wave emitter is positioned distally of at least one other shock wave emitter of the first shock wave emitter, the second shock wave emitter, and the third shock wave emitter on the emitter band. Optionally, the at least one shock wave emitter of the first shock wave emitter, the second shock wave emitter, and the third shock wave emitter is axially offset from the at least one other shock wave emitter of the first shock wave emitter, the second shock wave emitter, and the third shock wave emitter on the emitter band by 1 millimeter to 5 millimeters. Optionally, the at least one shock wave emitter of the first shock wave emitter, the second shock wave emitter, and the third shock wave emitter is axially offset from the at least one other shock wave emitter of the first shock wave emitter, the second shock wave emitter, and the third shock wave emitter on the emitter band by 1 millimeter to 3 millimeters. Optionally, the emitter band is at least 3 millimeters long.

Optionally, the emitter band comprises a proximal band portion, a distal band portion, and a connecting region between the proximal band portion and the distal band portion. Optionally, the connecting region comprises a bracket that connects the proximal band portion and the distal band portion, wherein the proximal band portion forms a cylindrical proximal band portion and the distal band portion forms a cylindrical distal band portion. Optionally, the emitter band comprises a plurality of openings positioned between at least one shock wave emitter of the first shock wave emitter, the second shock wave emitter, and the third shock wave emitter and at least one other shock wave emitter of the first shock wave emitter, the second shock wave emitter, and the third shock wave emitter. Optionally, the plurality of openings are positioned distally of the at least one shock wave emitter of the first shock wave emitter, the second shock wave emitter, and the third shock wave emitter and proximally of the at least one other shock wave emitter of the first shock wave emitter, the second shock wave emitter, and the third shock wave emitter. Optionally, the emitter band comprises a plurality of openings positioned between at least one shock wave emitter of the four shock wave emitters and at least one other shock wave emitter of the four shock wave emitters. Optionally, the plurality of openings are positioned distally of the at least one shock wave emitter of the four shock wave emitters and proximally of the at least one other shock wave emitter of the four shock wave emitters. Optionally, the plurality of openings are formed using a laser cutter.

Optionally, the emitter band comprises a plurality of thinned regions positioned between at least one shock wave emitter of the first shock wave emitter, the second shock wave emitter, and the third shock wave emitter and at least one other shock wave emitter of the first shock wave emitter, the second shock wave emitter, and the third shock wave emitter. Optionally, the plurality of thinned regions are positioned distally of the at least one shock wave emitter of the first shock wave emitter, the second shock wave emitter, and the third shock wave emitter and proximally of the at least one other shock wave emitter of the first shock wave emitter, the second shock wave emitter, and the third shock wave emitter. Optionally, the emitter band comprises a plurality of thinned regions positioned between at least one shock wave emitter of the four shock wave emitters and at least one other shock wave emitter of the four shock wave emitters. Optionally, the plurality of thinned regions are positioned distally of the at least one shock wave emitter of the four shock wave emitters and proximally of the at least one other shock wave emitter of the four shock wave emitters. Optionally, the enclosure is a balloon configured to be filled or inflated with a conductive fluid.

According to an aspect, an exemplary system for generating shock waves includes any of the catheters described above and a shock wave energy generator for generating energy pulses for driving shock wave emitters. The shock wave energy generator may generate energy pulses for driving at least three shock wave emitters. The pulse generator may comprise a switching circuit for driving at least one of the shock wave emitters independently of at least one other shock wave emitter of the at least three shock wave emitters. Optionally, the at least one shock wave emitter and the at least one other shock wave emitter are respectively connected to a first channel and a second channel of the switching circuit by a first supply wire and a second supply wire. Optionally, at least one of the at least three shock wave emitters is connected to a ground terminal of the switching circuit. Optionally, the shock wave energy generator is configured to deliver high voltage pulses to at least one of the at least three shock wave emitters.

According to an aspect, an exemplary method for treating a target area in a body lumen may include using any of the catheters described above and any of the systems described above. The method may include positioning a catheter in the body lumen adjacent to the target area, the catheter comprising an emitter band that forms at least three shock wave emitters; and driving at least one but not all of the at least three shock wave emitters so that shock waves are emitted from the at least one shock wave emitter and not from at least one other of the at least three shock wave emitters. Optionally, driving the at least one shock wave emitter causes at least two shock wave emitters of the emitter band to generate a respective shock wave. Optionally, the at least two shock wave emitters that generate a respective shock wave comprise a shock wave emitter connected to a negative terminal of a voltage source and a different shock wave emitter connected to ground. Optionally, driving the at least one shock wave emitter causes at least one other shock wave emitter of a second emitter band to generate a shock wave.

### BRIEF DESCRIPTION OF THE FIGURES

Illustrative aspects of the present disclosure are described in detail below with reference to the following figures. It is intended that the embodiments and figures disclosed herein are to be considered illustrative and exemplary rather than restrictive.

FIG. 1 illustrates an exemplary system for generating shock wave according to some embodiments.

FIG. 2A illustrates an isometric view of a portion of a catheter for generating shock waves according to some embodiments.

FIG. 2B illustrates a front view of an emitter band for generating shock waves according to some embodiments.

FIG. 2C illustrates a diagram of a portion of a catheter including three shock wave emitters according to some embodiments.

FIG. 3 illustrates a diagram of a portion of a catheter including four shock wave emitters according to some embodiments.

FIG. 4 illustrates a diagram of a portion of a catheter including two emitter bands according to some embodiments.

FIG. 5 illustrates a diagram of a portion of a catheter including two emitter bands according to some embodiments.

FIG. 6 illustrates a diagram of a portion of a catheter including two emitter bands according to some embodiments.

FIG. 7 illustrates a diagram of a portion of a catheter including three emitter bands according to some embodiments.

FIG. 8 illustrates an exemplary switching circuit and wiring schematic according to some embodiments.

FIG. 9 illustrates an exemplary system for generating shock waves according to some embodiments.

FIG. 10 illustrates an exemplary method for generating shock waves according to some embodiments.

FIG. 11 illustrates an isometric view of an emitter band including three apertures according to some embodiments.

FIG. 12 illustrates an isometric view of an emitter band including four apertures according to some embodiments.

FIG. 13 illustrates an exemplary emitter band with a plurality of apertures spaced non-uniformly about the emitter band according to some embodiments.

FIGS. 14A-14C depict emitter bands that include a plurality of openings, according to aspects of the disclosure.

FIG. 14D depicts another emitter band including a proximal band portion, a distal band portion, and a connecting region, according to aspects of the disclosure.

FIG. 14E illustrates how relatively longer emitter bands may be connected on a catheter, according to aspects of the disclosure.

FIG. 15 illustrates an exemplary computing device, according to aspects of the disclosure.

### DETAILED DESCRIPTION

The following description is presented to enable a person of ordinary skill in the art to make and use the various embodiments and aspects thereof disclosed herein. Descriptions of specific devices, assemblies, techniques, and applications are provided only as examples. Various modifications to the examples described herein will be readily apparent to those of ordinary skill in the art, and the general principles described herein may be applied to other examples and applications without departing from the spirit and scope of the various embodiments and aspects thereof. Thus, the various embodiments and aspects thereof are not intended to be limited to the examples described herein and shown but are to be accorded the scope consistent with the claims.

Described herein are systems, devices, and methods for generating shock waves at a plurality of circumferential locations using at least three shock wave emitters spaced around the circumference of a catheter. The catheters described herein may be used to treat hard-to-crack calcifications, notably those of eccentric and nodular morphologies or those found in large body lumens. The catheters described herein may be used to treat lesions (e.g., calcifications) in cavities and lumens such as those found in the vasculature, esophagus, trachea, Eustachian tube, and urinary tract. In accordance with the present disclosure, a catheter may include a catheter body and at least one emitter band positioned at least partially around the catheter body. At least three electrodes may be positioned adjacent to the emitter band and spaced apart from the emitter band by spark gaps, thus forming at least three shock wave emitters. The electrodes are positioned such that when a high voltage is applied across the spark gap separating a respective electrode from the emitter band, a shock wave is generated that propagates outwardly in a radial direction from the emitter band and catheter body.

The shock wave emitters can be configured so that at least two of the shock wave emitters formed by the emitter band can be driven independently of each other. For instance, a first electrode of the at least three shock wave emitters at the emitter band may be electrically connected to a first power supply channel of a voltage source and a second electrode of a different shock wave emitter may be connected to a second power supply channel that can be driven independently of the first power supply channel. A third electrode of the at least three electrodes may be connected to a ground terminal. Accordingly, pulsing either the first or second supply channel generates shock waves at two of the emitters-one at the shock wave emitter electrically connected to that supply channel and one at the shock wave emitter connected to the ground terminal. Pulsing the other supply channel also generates shock waves at two of the emitters but at a different set of the emitters-one at the shock wave emitter electrically connected to that supply channel and one at the shock wave emitter connected to the ground terminal. Pulsing either supply channel will not result in a shock wave generated at the shock wave emitter connected to the non-pulsed supply channel.

Put in different terms, the first emitter and the third emitter may be connected in series (thus forming a complete circuit between the first supply channel and the ground terminal). The second emitter and the third emitter may also be connected in series (thus forming a complete circuit between the first supply channel and the ground terminal). However, the first emitter and the second emitter may not be connected in series, enabling independent firing of the first and second emitters. That is, the first and second emitters may share a common ground but may be connected to different supply channels. It should be understood that “first,” “second,” and “third” as used here is an arbitrary notation. The first and third emitters may instead be connected to the two separate supply channels and thus not connected in series. Any number of shock wave emitters electrically connected to any number of supply channels on a single emitter band and/or on more than one emitter band, may be driven independently from one another and in series with a shock wave emitter connected to a ground terminal as described.

The emitter band may include apertures, each of which may form a portion of a different shock wave emitter. The apertures formed in the emitter band may be spaced from one another about the circumference of the emitter band to enable generation of shock waves that propagate in at least three different directions outwardly from the emitter band. For instance, the apertures may be evenly distributed about the circumference of the emitter band (e.g., 120-degrees apart when the emitter band includes three apertures or 90-degrees apart when the emitter band includes four apertures), or the apertures may be distributed unevenly (e.g., irregularly dispersed about the emitter band). The apertures, and thus the shock wave emitters, may be spaced apart from one another in a circumferential direction about the emitter band by any of 60 degrees, 72 degrees, 90 degrees, or 120 degrees. The apertures, and thus the shock wave emitters, may be spaced apart from one another in a circumferential direction about the emitter band by a range of 60 degrees to less than 180 degrees (e.g., up to 72 degrees, up to 90 degrees, up to 120 degrees, up to 135 degrees, or up to 179 degrees). In some examples, apertures, and thus the shock wave emitters, may be spaced apart from one another in a circumferential direction about the emitter band by any angle between 15 degrees and 180 degrees.

The apertures in emitter bands that form a portion of the shock wave emitters described herein may be shaped as circles, ovals, triangles, squares, rectangles, or other polygons. In some examples, when apertures are shaped into polygonal apertures, the apertures may include rounded corners (e.g., fillets). Upon application of high voltage pulses, mechanical stress may become concentrated at sharp corners causing the emitter bands to crack. Accordingly, rounded corners may mitigate damage to the emitter bands during shock wave generation. One or more of the emitter bands and/or regions of the emitter bands including the apertures may be reinforced with additional conductive material, materials having relatively higher durability, and/or providing additional space between the emitter band and the electrode(s) adjacent to the emitter band within the apertures. In some examples, one or more of the electrodes positioned adjacent to the emitter band(s) may additionally, or alternatively, be reinforced against erosion by using a relatively more durable material (e.g., molybdenum or tungsten instead of copper). For instance, in some examples, an electrode connected to a ground channel may be utilized more often in shock wave generation because it may be used to generate a shock wave whenever any supply channel is pulsed. Accordingly, the electrode connected to ground, or the region of the emitter band adjacent to the electrode connected to ground (e.g., the material forming the aperture), may be reinforced against erosion by using a relatively more durable material.

The catheters disclosed herein may be particularly useful for treating hard to crack calcifications, such as those of nodular and/or eccentric morphologies, due to the ability to generate shock waves in at least three directions outwardly from the emitter band without requiring rotation of the catheters within the vasculature, which can be impractical in clinical settings because a surgeon may have no way to determine which direction the emitters are directed when the catheter is inserted into a patient. The catheters having closely spaced shock wave emitters (circumferentially and/or axially) may be beneficial for targeting lesions of large body lumens (e.g., blood vessels having a diameter no less than 1.0 cm or structural heart lumens), for instance due to increased constructive interference between the closely spaced shock wave emitters, as described further below.

Positioning shock wave emitters relatively closer together around the circumference of the catheter body (e.g., at 60-degree increments, 90-degree increments, 120-degree increments, etc.) may result in increased sonic output at a location between the two emitters due to increased constructive interferences relative to 180-degree spacing between the emitters. Additionally, providing more than two shock wave emitters at an emitter band location can offset the increased distance (i.e., “shadow-zone”) that may result in by positioning two shock wave emitters such that less than 180-degrees separate the two emitters on one side of the catheter and more than 180-degrees separate the emitters on the other side of the catheter. The emitter band and shock wave emitters may be enclosed within an enclosure that may be filled with a conductive fluid that enables sparks to generate across the spark gaps.

The catheters described herein can be inserted into a body lumen, for instance, to treat a buildup of calcification. The catheter may be advanced within the body lumen until one or more of the emitter bands positioned on the catheter are positioned at a desired distance (e.g., adjacent to) the target treatment area. Once in position, a plurality of shock waves may be emitted from the shock wave emitters such that the shock waves propagate outwardly in a plurality of directions toward the target treatment area. By spacing at least three emitters about the circumference of the emitter band, the catheters described herein can treat nodular and eccentric calcifications more effectively than conventional catheters having only two emitters at any given longitudinal location by enabling generation of shock waves in at least three directions without rotating the catheter. The shock waves may constructively interfere with one another outwardly of the catheter, thus compounding the peak compressive force of the plurality of shock waves relative to each of the individual shock waves emitted by the respective shock wave emitters. Sonic output may thus be enhanced via the positioning of shock wave emitters relatively close together axially and/or circumferentially, without requiring higher voltages/increased energy supply. In some embodiments, sonic output from constructively interfering acoustic waves generated from shock wave emitters circumferentially spaced less than 180 degrees from each other may be at least 1.0 megapascal (MPa) higher at a treatment site (e.g., a calcified lesion) than sonic output from acoustic waves generated from shock wave emitters circumferentially spaced 180 degrees from each other using the same energy source (for instance, a 3 kV voltage source). For example, constructively interfering acoustic waves may apply at least 5 MPa of pressure at a treatment site. In some embodiments, constructively interfering acoustic waves may apply at least 6 MPa of pressure at a treatment site. After breaking up a portion of the target treatment area (e.g., a portion of the calcification/occlusion), the catheter may be advanced further into the vessel toward a second target treatment area, and a second plurality of shock waves may be emitted targeting the treatment area. This process may be iterated any number of times until the calcification has been successfully treated.

Efforts have been made to improve the design of electrode assemblies included in shock wave and directed cavitation catheters. For instance, low-profile electrode assemblies have been developed that reduce the crossing profile of a catheter and allow the catheter to more easily navigate calcified vessels to deliver shock waves in more severely occluded regions of vasculature. Examples of low-profile electrode designs can be found in U.S. Pat. Nos. 8,888,788, 9,433,428, and 10,709,462, and in U.S. Publication No. 2021/0085383 all of which are incorporated herein by reference. Other catheter designs have improved the delivery of shock waves, for instance, by specific electrode construction and configuration thereby directing shock waves in a forward direction to break up tighter and harder-to-cross occlusions in vasculature. Examples of forward-firing catheter designs can be found in U.S. Pat. Nos. 10,966,737, 11,478,261, and 11,596,423 and U.S. Publication Nos. 2023/0107690 and 2023/0165598, all of which are incorporated herein by reference. Other catheter designs have sought to take advantage of constructive interference of propagating shock waves by controlling the longitudinal and/or circumferential spacing of shock wave emitters or emitter bands. An example of catheter designs with interfering shock waves may be found in U.S. Pat. No. 11,779,363, which is incorporated herein by reference.

As used herein, the term “electrode” refers to an electrically conducting element (typically made of metal) that receives electrical current and subsequently releases the electrical current to another electrically conducting element. In the context of the present disclosure, electrodes are often positioned relative to each other, such as in an arrangement of an inner electrode and an outer electrode. Accordingly, as used herein, the term “electrode pair” refers to two electrodes that are positioned adjacent to each other such that application of a sufficiently high voltage to the electrode pair will cause an electrical current to transmit across the gap (also referred to as a “spark gap”) between the two electrodes (e.g., from an inner electrode to an outer electrode, or vice versa, optionally with the electricity passing through a conductive fluid or gas therebetween). In some contexts, one or more electrode pairs may also be referred to as an electrode assembly. In the context of the present disclosure, the term “emitter” broadly refers to the region of an electrode assembly where the current transmits across the electrode pair, generating a shock wave. The terms “emitter sheath” and “emitter band” refers to a continuous or discontinuous band of conductive material that may form one or more electrodes of one or more electrode pairs, thereby forming a location of one or more emitters.

Components of emitters, including electrodes and emitter sheaths/bands, may be formed from a metal, such as stainless steel, copper, tungsten, platinum, palladium, molybdenum, cobalt, chromium, iridium, an alloy or alloys thereof, such as cobalt-chromium, platinum-chromium, cobalt-chromium-platinum-palladium-iridium, or platinum-iridium, or a mixture of such materials.

For treatment of an occlusion in a blood vessel, the voltage pulse applied by a power source, including any of the power sources described herein (which may also be referred to herein as voltage sources or pulse generators), is typically in the range of from about five hundred to three thousand volts (500 V-3,000 V). In some implementations, the voltage pulse applied by the voltage source can be up to about ten thousand volts (10,000 V) or higher than ten thousand volts (10,000 V). The pulse width of the applied voltage pulses ranges between two microseconds and six microseconds (2-6 μs). The repetition rate or frequency of the applied voltage pulses may be between about 1 Hz and 10 Hz. The total number of pulses applied by the power source may be, for example, sixty (60) pulses, eighty (80) pulses, one hundred twenty (120) pulses, three hundred (300) pulses, or up to five hundred (500) pulses, or any increments of pulses within this range. Alternatively, or additionally, in some examples, the power source may be configured to deliver a packet of micro-pulses having a sub-frequency between about 100 Hz-10 kHz. The preferred voltage, repetition rate, and number of pulses may vary depending on, e.g., the size of the lesion, the extent of calcification, the size of the blood vessel, the attributes of the patient, or the stage of treatment. For instance, a physician may start with low energy shock waves and increase the energy as needed during the procedure, or vice versa. The magnitude of the shock waves can be controlled by controlling the voltage, current, duration, and repetition rate of the pulsed voltage from the power source.

In some embodiments, an IVL catheter is a so-called “rapid exchange-type” (“Rx”) catheter provided with an opening portion through which a guide wire is guided (e.g., through a middle portion of a central tube in a longitudinal direction). In other embodiments, an IVL catheter may be an “over-the-wire-type” (“OTW”) catheter in which a guide wire lumen is formed throughout the overall length of the catheter, and a guide wire is guided through the proximal end of a hub.

Although shock wave devices described herein generate shock waves based on high voltage applied to electrodes, it should be understood that a shock wave device additionally or alternatively may comprise a laser and optical fibers as a shock wave emitter system whereby the laser source delivers energy through an optical fiber and into a fluid to form shock waves and/or cavitation bubbles.

In the following description of the various embodiments, reference is made to the accompanying drawings, in which are shown, by way of illustration, specific embodiments that can be practiced. It is to be understood that other embodiments and examples can be practiced, and changes can be made without departing from the scope of the disclosure.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure in some embodiments also relates to a device for performing the operations herein. This device may be specially constructed for the required purposes, or it may comprise a general-purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each connected to a computer system bus. Furthermore, the computing systems referred to in the specification may include a single processor or may be architectures employing multiple processor designs, such as for performing different functions or for increased computing capability. Suitable processors include central processing units (CPUs), graphical processing units (GPUs), field programmable gate arrays (FPGAs), and ASICs.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The structure for a variety of these systems will appear from the description below. In addition, the present invention is not described with reference to any particular programming language. It will be appreciated that a variety of programming languages may be used to implement the teachings of the present disclosure as described herein.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof. As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement.

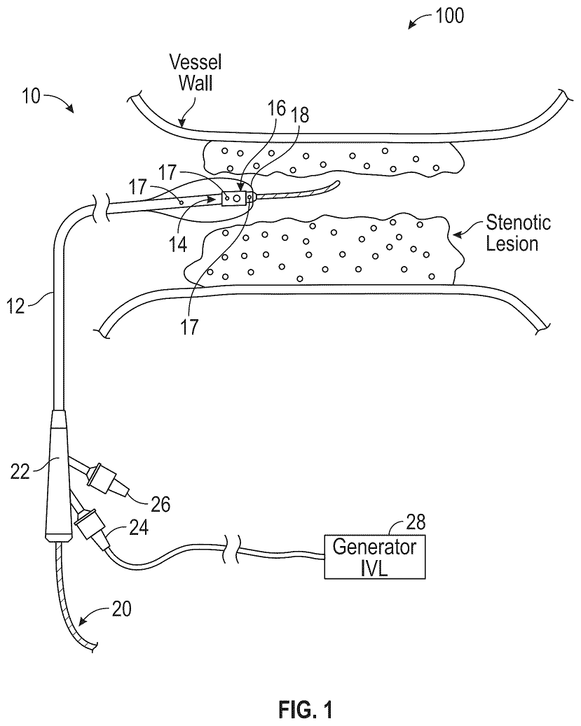

FIG. 1 illustrates a system 100 for treating calcifications in body lumens. The system includes a shock wave generating catheter 10. The catheter 100 may be used to fragment, crack, or otherwise break up calculi within a body lumen, for instance, to treat various occlusions within blood vessels. The catheter 10 includes at its distal end a plurality of shock wave emitters 16 positioned within an enclosure 18. The catheter 10 is advanced into an occlusion in a patient's vasculature, such as the stenotic lesion depicted in FIG. 1, over a guidewire 20 carried in a guidewire sheath and voltage pulses are applied to the shock wave emitters 16 to generate shock waves. As used herein, each of the one or more emitters include electrode pairs having first and second electrodes separated by a gap, at which shock waves are formed when a current flows across the gap between the electrodes of the pair (i.e., when a voltage is applied across the first and second electrodes). The electrode pairs described herein may be formed by an emitter band and a plurality of electrodes positioned adjacent to the emitter band. Each electrode may form a respective electrode pair with the emitter band.

An enclosure 18 (e.g., a low-profile flexible angioplasty balloon, a polymer membrane in tension that can flex outward, etc.) is sealably attached to the distal end 14 of the catheter 10, forming an annular channel around the shaft 12 of the catheter. The enclosure 18 surrounds the plurality of shock wave emitters 16, such that the shock waves are produced in a closed system within the enclosure 18. The enclosure 18 is filled or inflated with a conductive fluid, such as saline. The enclosure 18 can alternatively be referred to as a “window”, in particular for implementations where when the interior volume is filled with a fluid and pressurized, the window maintains a substantively constant volume and profile. The conductive fluid allows the acoustic shock waves to propagate outwardly from the electrode pairs of the shock wave generator 16 through the walls of the enclosure 18 and then into the target lesion. In one or more examples, the conductive fluid may also contain x-ray contrast fluid for fluoroscopic viewing of the catheter 10 during use. In some implementations, the material that forms the primary surface(s) of the enclosure 18 through which shock waves pass can be a noncompliant polymer. In other implementations, a rigid and inflexible structure may be used in lieu of enclosure 18. The enclosure 18 may mitigate thermal injury to soft tissue and reduce cavitation stresses by limiting expansion of the vapor bubbles produced during shock wave generation to the interior of the enclosure. For instance, the vapor bubbles hit the enclosure wall before reaching their maximum potential size, thus inducing collapse, and reducing cavitation stress and preventing soft tissue injury that can be caused by tensile stresses during cavitation bubble collapse.

The catheter 10 includes a proximal end 22 (or handle) that remains outside of a patient's vasculature during treatment. The proximal end 22 includes an entry port for receiving the guidewire 20. The proximal end 22 also includes a fluid port 26 for receiving a conductive fluid for filling and emptying the enclosure 18 during treatment. An electrical connection port 24 is also located on the proximal end 22 to provide an electrical connection between the distal shock wave emitters 16 and an external pulsed high voltage source 28, such as the intravascular lithotripsy (IVL) generator shown in FIG. 1.

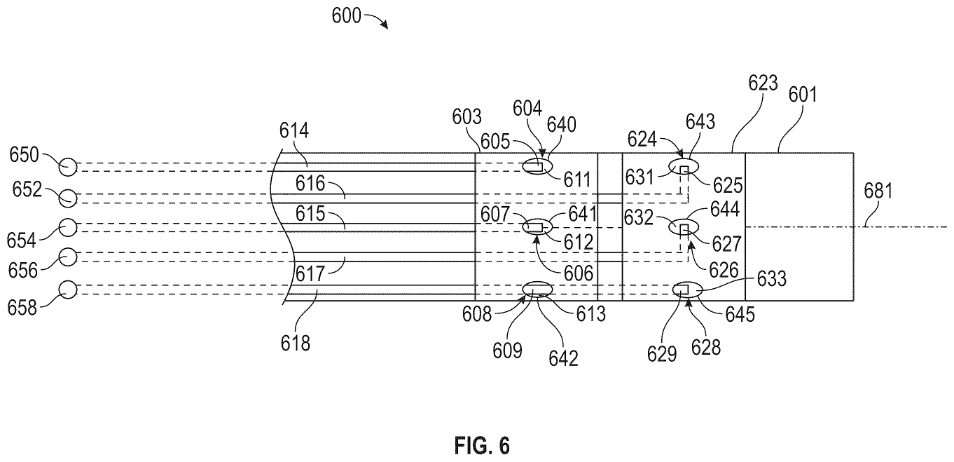

The catheter 10 also includes a flexible shaft 12 that extends from the proximal end 22 to the distal end 14 of the catheter. The shaft 12 provides various internal conduits connecting elements of the distal end 14 with the proximal end 22 of the catheter (see, e.g., FIG. 6D for a cross-section of a region an exemplary shaft). The shaft 12 includes an elongate tube that includes a lumen for receiving the guidewire 20. The elongate tube may include additional lumens extending through the shaft 12 or along an outer surface of the shaft 12. For example, one for fluid lumens (e.g., a fluid inlet lumen and a fluid outlet lumen or a combined flush lumen) can be located along or within the shaft 12 for carrying conductive fluid from the fluid port 26 into the enclosure 18.

In some examples, one or more sensors 17 are positioned along the catheter 10. The sensors 17 may be positioned at any location on catheter 10. For instance, the sensors 17 may be positioned proximal to one or more shock wave emitters 16, distal from one or more shock wave emitters 16, and/or intermediary between one or more shock wave emitters 16 (or any combination thereof). The sensors 17 may be positioned external to the enclosure 18 and/or outside of a patient. For instance, certain sensors, such as a pressure sensor, may be positioned outside of the enclosure 18 and/or the patient because the pressure may be measured on the system as a whole when components are in fluid communication. The sensors may include one or more of any suitable sensor devices, such as a pressure sensor, a thermal sensor, an electrical sensor (e.g., current, voltage, resistance, and/or impedance sensors), or a visualization element. Sensors 17 can provide feedback to an operator using catheter 10 by measuring parameters in the surrounding environment and thereby indicating a status of the catheter 10 and components thereof, and further providing for guidance on what further steps the operator may decide to implement with catheter 10. For example, in implementations where sensor devices 17 include pressure sensors, a slight decrease in pressure may indicate success at cracking a calcified lesion, due to the fact that the expandable member surrounding the emitters is able to further expand without changing the volume of fluid within the expandable member. Further, a significant decrease in pressure may indicate a rupture failure mode where the expandable member has lost seal and fluid volume, and thus guiding toward withdrawal of the device. In implementations where the sensor devices include a visualization element, an operator of the catheter 10 may be able to more clearly understand where the catheter device 10 is located relative to a target lesion or anatomy, prior to, during, and after delivering therapy.

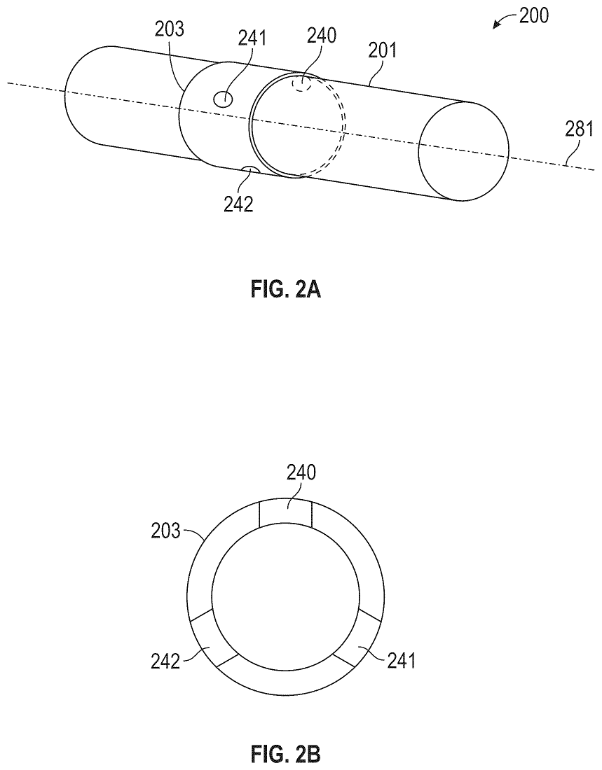

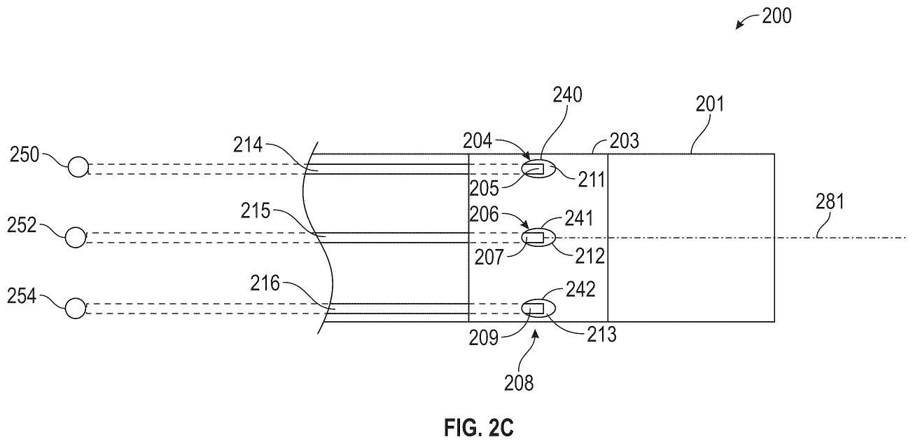

FIGS. 2A-2C illustrate an exemplary catheter 200 that can be used for the catheter 10 of FIG. 1. The catheter 200 includes a catheter body 201 and a shock wave emitter band 203 that forms a portion of multiple shock wave emitters that may be positioned on the catheter body 201. FIG. 2A illustrates an isometric view of catheter 200 and shock wave emitter band 203. The emitter band 203 may be a conductive cylindrical band extending around the catheter body 201 and may be configured to enable generation of shock waves at a plurality of different circumferential locations to treat eccentric and/or nodular calcifications. The emitter band 203 includes three apertures 240, 241, and 242. The apertures are spaced from one another in the circumferential direction about a longitudinal axis 281.

As illustrated in the cross-sectional view of FIG. 2B, the apertures 240, 241, and 242 are equally spaced 120 degrees from one another and extend through a thickness of the emitter band, thus forming an opening through the emitter band from the outer surface to the inner surface. The emitter band 203 forms one of the electrodes of the electrode pair of each shock wave emitter. The other electrode of each electrode pair may be formed by a respective electrode that is positioned adjacent to the respective aperture of emitter band 203, as described below with reference to FIG. 2C.

FIG. 2C illustrates an example of shock wave catheter 200 that depicts an arrangement of electrodes 205, 207, and 209 relative to emitter band 203 (the view of FIG. 2C depicts the emitter band as if it were cut and laid out flat). Each of the electrodes 205, 207, and 209, together with the emitter band 203, forms an electrode pair of a shock wave emitter. For instance, electrode 205 and emitter band 203 may form one electrode pair of one shock wave emitter, and electrode 206 and emitter band 203 may form another electrode pair of another shock wave emitter. The electrodes 205, 206, and 207 may be formed by ends of respective conductive elements 214, 215, and 216. In the illustrated example, the conductive elements are insulated wires that have uninsulated ends. The conductive elements (e.g., insulated wires) 214, 215, and 216 may extend along the length of the catheter 200 to connect to a respective supply channel or ground terminal, as described further below. In some examples, the electrodes 205, 206, and 207 may each be formed by a metallic surface (e.g., of a metallic sleeve) that is electrically connected to one of the conductive elements 214, 215, and 216.

Electrode 205 may be positioned radially inwardly of the emitter band 203, with its distal end positioned adjacent to aperture 240. Similarly, electrode 207 may be positioned radially inwardly of the emitter band 203, with its distal end positioned adjacent to aperture 241, and electrode 209 may be positioned radially inwardly of the emitter band 203, with its distal end positioned adjacent to aperture 242. The electrodes 205, 207, and 209 are spaced apart from the emitter band 203 by a respective spark gap 211, 212, and 213 resulting from the respective apertures, thus forming a plurality of shock wave emitters 204, 206, and 208 positioned about the circumference of catheter body 201 for generating shock waves.

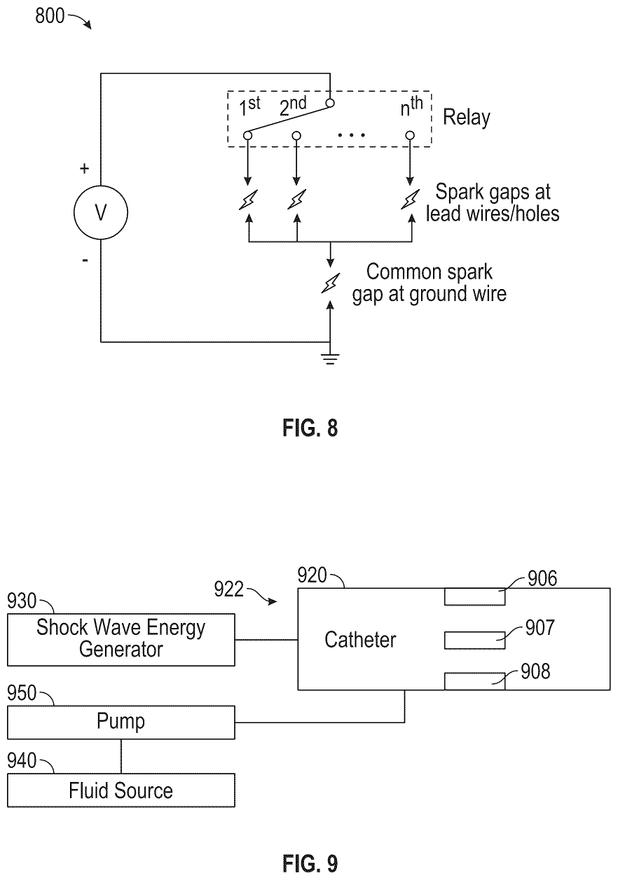

At least one of the shock wave emitters may be configured to be driven independently of at least one of the other shock wave emitters. A proximal end of conductive element 214 may be electrically connected to a first supply channel 250 (for instance, a supply channel of switching circuit 800 illustrated in FIG. 8), thus electrically connecting electrode 205 to supply channel 250. A proximal end of conductive element 215 may be electrically connected to a second supply channel 252, thus also electrically connecting electrode 207 to supply channel 250. Finally, a proximal end of conductive element 216 may be electrically connected to a ground terminal 254, thus electrically connecting electrode 209 to ground terminal 254. Accordingly, when a sufficiently high voltage (e.g., pulse, voltage differential) is applied (e.g., by shock wave energy generator 930 of FIG. 9) across the ground terminal 254 and supply channel 150, a shock wave is generated at both emitter 208 and emitter 204 simultaneously. When a sufficiently high voltage pulse is applied across the ground terminal 254 and supply channel 252, a shock wave is generated at both emitter 208 and emitter 206, simultaneously.

More specifically, supplying one or more suitably high voltage pulses to the first channel 250 will cause one or more shock waves to be generated at shock wave emitter 204 as a current flows across the spark gap 211 between electrode 205 and emitter band 203, and supplying one or more suitably high voltage pulses to the second channel 252 will cause a shock wave to be generated at shock wave emitter 206 as a current flows across the spark gap 212 between electrode 207 and emitter band 203. Shock wave emitter 204 may be driven independently of shock wave emitter 206 by applying one or more voltage pulses to the first channel 250, and shock wave emitter 206 may be driven independently of shock wave emitter 206 by applying one or more voltage pulses to the second channel 252. However, applying one or more voltage pulses to either the first channel 250 or second channel 252 will also cause a shock wave to be simultaneously generated at emitter 208 as the current flows across spark gap 213 between electrode 209 and emitter band 203, following the path to ground.

Thus, shock wave emitters 204 and 206 can be driven independently of one another but generating a shock wave at either emitter 204 or 206 will result in generation of at least two shock waves because shock wave emitter 204 and shock wave emitter 206 are respectively connected in series with shock wave emitter 208. It should be understood that any two of the shock wave emitters 204-208 could be connected to the first and second supply channel 250 and 252 of the switching circuit, and any of the shock wave emitters 204-208 could be connected to ground terminal 254. As described, the ability to generate shock waves originating from at least three circumferential locations about a given emitter band enables the catheters described herein to effectively treat relatively harder to crack calcifications, such as nodular and eccentric calcifications, relatively denser calcifications, calcifications positioned further from the shock wave emitters, and/or calcifications having different and/or irregular calcific morphologies within a body lumen. Further, additional shock wave emitters may be connected to additional corresponding channels, as described further throughout.

The conductive elements 214, 215, and 216 may be disposed in cavities in an outer wall of the catheter body 201 and/or within internal lumens extending along the length of the catheter body to connect to a voltage source (e.g., shock wave energy generator 930). Any of electrodes 205, 207, and 209 may additionally or alternatively include a conductive material (e.g., a conductive sheath) soldered, clamped, welded, or otherwise electrically connected to a conductive portion of a respective conductive element 214, 215, or 216. Electrodes 205, 207, and 209 may be the same material as the respective conductive elements 214, 215, and 216 or may be formed at least partially of a different material from the respective conductive elements 214, 215, and 216. For example, one or more of the electrodes 205, 207, and 209 may be formed from molybdenum, tungsten, or other more durable materials relative to, for instance, copper, as described in U.S. patent application Ser. No. 18/586,299 and U.S. patent application Ser. No. 18/680,370, which are each incorporated herein by reference in their entirety for all purposes.

While FIGS. 2A-2C depict a catheter with a single emitter band that forms an electrode of three shock wave emitters, it should be understood that additional electrodes may be paired with the emitter band to form additional shock wave emitters, and/or additional emitter bands and corresponding electrodes forming shock wave emitters may be positioned along the length of the catheter body such that the catheters disclosed herein can be used to treat multiple regions of calcified buildup simultaneously. In some examples, relatively larger and/or relatively smaller diameter emitter bands may be implemented in any of the examples described herein. It may be relatively easier to fit additional apertures (assuming the aperture diameter remains constant) on larger emitter bands than smaller emitter bands. In some examples, relatively thicker and/or relatively thinner emitter bands may be implemented in any of the examples described herein. In some examples, relatively thicker emitter bands (or materials made from relatively durable material, such as 316SS) will have more material to erode or material that is more difficult to erode, resulting more pulses before shock wave emitter failure. In some examples, emitter bands may be positioned on an insulation layer, and in some examples, relatively thicker bands may utilize a relatively thicker layer of insulation. Relatively thicker and/or larger diameter emitter bands, however, may be more difficult to navigate within smaller body lumens. Accordingly, in some examples, relatively smaller emitter bands may provide for a more navigable device within the body.

In some examples, any of the emitter bands described herein (e.g., band 203) may have an outer diameter between 0.025 inches and 0.125 inches. In some examples, any of the emitter bands described herein (e.g., band 203) may have an outer diameter between 0.034 inches and 0.108 inches. In some examples, any of the emitter bands described herein (e.g., band 203) may have an outer diameter between 0.034 inches and 0.0565 inches. In some examples, any of the emitter bands described herein (e.g., band 203) may have an outer diameter of at least 0.025 inches, at least 0.035 inches, at least 0.045 inches, at least 0.055 inches, at least 0.065 inches, at least 0.075 inches, at least 0.085 inches, at least 0.095 inches, at least 0.105 inches, at least 0.115 inches, and/or at least 0.125 inches. In some examples, any of the emitter bands described herein (e.g., band 203) may have an outer diameter of at most 0.025 inches, at most 0.035 inches, at most 0.045 inches, at most 0.055 inches, at most 0.065 inches, at most 0.075 inches, at most 0.085 inches, at most 0.095 inches, at most 0.105 inches, at most 0.115 inches, and/or at most 0.125 inches.

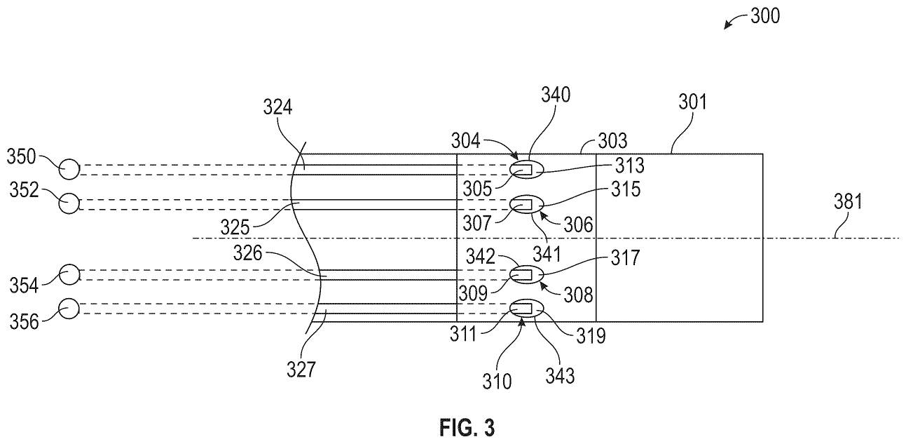

FIGS. 3-7, described in further detail below, illustrate catheters that include additional shock wave emitters and/or multiple emitter bands and corresponding shock wave emitters along the length of the catheter body. FIG. 3 illustrates a schematic of a catheter 300 with four electrodes positioned adjacent to four apertures of an emitter band, thus forming four shock wave emitters according to some embodiments (the view of FIG. 3 depicts the emitter band as if it were cut and laid out flat). The catheter 300 may include any of the features described above with reference to catheter 100 and 200 of FIGS. 1-2C along with an additional (i.e., fourth) shock wave emitter positioned at the emitter band.

The catheter 300 includes a catheter body 301 and an emitter band 303 positioned on the catheter body 301. Emitter band 303 forms a portion of multiple shock wave emitters that may be positioned on the catheter 300 for generating shock waves. Like emitter band 203, emitter band 303 may be a conductive cylindrical band extending around the catheter body 301. Emitter band 303, however, includes four apertures 340, 341, 342, and 343. The apertures are spaced from one another in the circumferential direction about longitudinal axis 381 of catheter body 301. The apertures may be equally spaced apart 90 degrees from one another about longitudinal axis 381 and may extend through a thickness of the emitter band 303. Emitter band 303 forms one electrode of each electrode pair that corresponds to a respective shock wave emitter. The other electrode of each pair each electrode pair may be formed by a respective electrode that is positioned adjacent to the respective aperture of emitter band 303.

As shown, four electrodes 305, 307, 309, and 311 are arranged adjacent to emitter band 303. Each of the respective electrodes 305, 307, 309, and 311 form one electrode of the four electrode pairs completed by emitter band 303. For instance, electrode 305 and emitter band 303 may form a first electrode pair, electrode 307 and emitter band 303 may form a second electrode pair, and so on. Electrodes 305, 307, 309, and 311 may be formed by uninsulated ends of respective conductive elements 324, 325, 326, and 327. The conductive elements 324, 325, 326, and 327 may be insulated conductive wires (e.g., copper wires, molybdenum wires, tungsten wires, tantalum wires). The conductive elements (e.g., insulated wires) 324, 325, 326, and 327 may extend along the length of the catheter 300 to connect to a respective supply channel or ground terminal, as described further below.

The conductive elements 324, 325, 326, and 327 may be disposed in cavities in an outer wall of the catheter body 301 and/or within internal lumens extending along the length of the catheter body to connect to a voltage source (e.g., shock wave energy generator 930). Any of electrodes 305, 307, 309 and 311 may additionally or alternatively include a conductive material (e.g., a conductive sheath) soldered, clamped, welded, or otherwise electrically connected to a conductive portion of a respective insulated wire 324, 325, 326, and 327. Electrodes 305, 307, 309, and 311 may be the same material as the respective conductive elements 324, 325, 326, and 327 or may be formed at least partially of a different material from the respective conductive elements 324, 325, 326, and 327.

Each of electrodes 305, 307, 309, and 311 may be positioned to align with one of the respective apertures 340, 341, 342, and 343. Electrode 305 may be positioned radially inwardly of the emitter band 303, with its distal end positioned adjacent aperture 340. Similarly, electrode 307 may be positioned radially inwardly of the emitter band 303, with its distal end positioned adjacent to aperture 341, electrode 309 may be positioned radially inwardly of the emitter band 303, with its distal end positioned adjacent aperture 342, and electrode 311 may be positioned radially inwardly of the emitter band 303, with its distal end positioned adjacent aperture 343. The electrodes 305, 307, and 309 are spaced apart from the emitter band 303 by a respective spark gap 313, 315, 317 and 319 resulting from the respective apertures, thus forming a plurality of shock wave emitters 304, 306, 308 and 310 positioned about the circumference of catheter body 301 for generating shock waves.

A proximal end of each of the conductive elements 324, 325, 326, and 327 may be electrically connected to a respective supply channel or ground terminal, and at least one of the shock wave emitters may be configured to be driven independently of at least one of the other shock wave emitters. A proximal end of conductive element 324 may be electrically connected to a first supply channel 350 (for instance, a supply channel of switching circuit 800 illustrated in FIG. 8), thus electrically connecting electrode 305 to supply channel 350. A proximal end of conductive element 325 may be electrically connected to a second supply channel 352, thus electrically connecting electrode 307 to supply channel 352. A proximal end of conductive element 326 may be electrically connected to a third supply channel 354, thus electrically connecting electrode 309 to supply channel 354. Finally, a proximal end of conductive element 327 may be electrically connected to a ground terminal 356, thus electrically connecting electrode 309 to ground terminal 356.

Accordingly, when a sufficiently high voltage (e.g., pulse, voltage differential) is applied (e.g., by shock wave energy generator 930 of FIG. 9) across the ground terminal 356 and any of the supply channels 350, 352, or 354, at least two shock waves are generated. For instance, when a sufficiently high voltage is applied across supply channel 350 and ground terminal 356, a shock wave is generated at both emitter 304 and emitter 310 simultaneously. When a sufficiently high voltage is applied across the supply channel 352 and ground terminal 356, a shock wave is generated at both emitter 306 and emitter 310, simultaneously. Finally, when a sufficiently high voltage is applied across the supply channel 354 and ground terminal 356, a shock wave is generated at both emitter 308 and emitter 310, simultaneously. In some examples, a sufficiently high voltage may be at least 0.40 kV per electrode pair, at least 0.45 kV per electrode pair, at least 0.50 kV per electrode pair, at least 0.55 kV per electrode pair, and/or at least 0.60 kV per electrode pair. In some embodiments, a voltage as high as 1.0 kV per electrode pair may be applied across each electrode pair. In some embodiments, a voltage as high as 1.5 kV per electrode pair may be applied across each electrode pair. In some embodiments, a voltage as high as 2.0 kV per electrode pair may be applied across each electrode pair.

More specifically, supplying one or more suitably high voltage pulses to the first supply channel 350 will cause a shock wave to be generated at shock wave emitter 304 as a current flows across the spark gap 313 between electrode 305 and emitter band 303, supplying one or more suitably high voltage pulses to the second channel 352 will cause a shock wave to be generated at shock wave emitter 306 as a current flows across the spark gap 315 between electrode 307 and emitter band 303, and supplying one or more suitably high voltage pulses to the third channel 354 will cause a shock wave to be generated at shock wave emitter 308 as a current flows across the spark gap 317 between electrode 309 and emitter band 303. Additionally, supplying one or more suitably high voltage pulses to any of the supply channels 305-354 will cause a shock wave to be generated at shock wave emitter 310 as a current flows across the spark gap 319 between electrode 311 and emitter band 303.

Thus, shock wave emitter 304 may be driven independently of shock wave emitters 306 and 308 by supplying one or more suitably high voltage pulses to the first channel 350, shock wave emitter 306 may be driven independently of shock wave emitters 304 and 308 by supplying one or more suitably high voltage pulses to the second channel 352, and shock wave emitter 308 may be driven independently of shock wave emitters 304 and 306 by supplying one or more suitably high voltage pulses to supply channel 354. However, supplying one or more suitably high voltage pulses to any of the first channel 350, second channel 352, or third channel 354 will also cause a shock wave to be simultaneously generated at emitter 310. Accordingly, generating a shock wave at any of emitters 304, 306, or 310 will result in generation of at least two shock waves because shock wave emitter 304, 306, and 310 are respectively connected in series with shock wave emitter 310. It should be understood that any of the shock wave emitters 304-308 could instead be connected to ground terminal 356. Further, additional shock wave emitters may be connected to additional corresponding channels, as described further throughout.

Accordingly, catheter 300 includes four shock wave emitters, and supplying one or more suitably high voltage pulses to any one of the three power supply channels will result in generation of at least two shock waves, one at the emitter connected to a supply channel and one at the emitter connected to ground. Thus, catheter 300 may enable generation of shock waves at four different circumferential locations about a longitudinal axis 381 of catheter 300 for treating nodular and/or eccentric calcifications without rotating the catheter. One having skill in the art would understand that any of the supply channels may serve as the ground terminal and the ground terminal could serve as a replacement supply channel, which may alter the flow of current through the conductive elements of the catheter. That is, changes to the electrical configuration of the catheter are possible without deviating from the scope of the disclosure.

In some embodiments, the apertures 340, 341, 342, and 343 of emitter band 303 are positioned to enhance constructive interference of propagating shock waves. For example, apertures 340 and 341 may be positioned to form a first pair of shock wave emitters and apertures 342 and 343 may be positioned to form a second pair of shock wave emitters. Apertures of each pair of emitters may be positioned closer to each other than apertures of the other pair of emitters (i.e., apertures of pairs of emitters are located less than 90 degrees away from each other about axis 381).

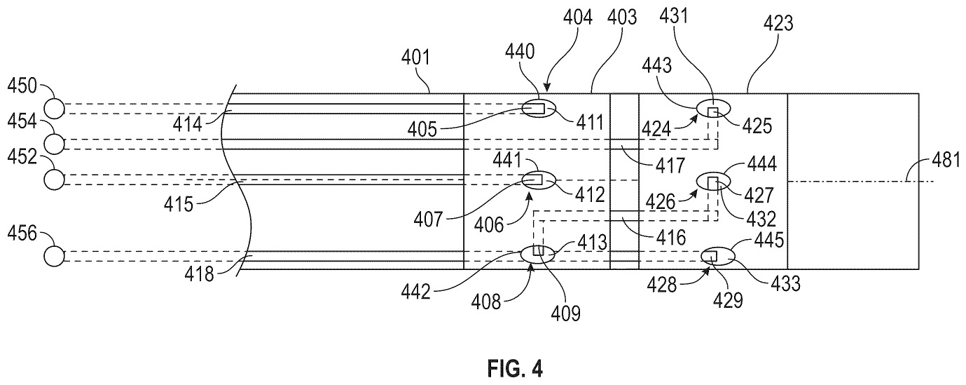

As noted above, a catheter can have more than one emitter band. FIG. 4 illustrates an example that includes at least two emitter bands, each forming an electrode of three shock wave emitters (the view of FIG. 4 depicts the emitter band as if it were cut and laid out flat). As shown, catheter 400 includes a catheter body 401 and two conductive emitter bands 403 and 423 positioned at two different locations (emitter band 423 positioned distally of emitter band 403) on the catheter body 401. Each of emitter bands 403 and 423 form a portion of multiple shock wave emitters that may be positioned on catheter 400 for generating shock waves. Emitter bands 403 and 423 may be conductive cylindrical bands extending around the catheter body 401. Emitter band 403 and 423 each include a respective set of apertures. Emitter band 403 includes apertures 440, 441, and 442. Emitter band 423 includes apertures 443, 444, and 445. The apertures on emitter band 403 are spaced from one another in the circumferential direction about longitudinal axis 481 of catheter body 401. Similarly, the apertures on emitter band 423 are spaced from one another in the circumferential direction about longitudinal axis 481 of catheter body 401. The apertures on each of emitter bands 403 and 423 may be equally spaced apart 120 degrees from one another about longitudinal axis 481 and may extend through a thickness of the respective emitter bands 403 and 423. Emitter bands 403 and 423 respectively form one electrode of each electrode pair that corresponds to a respective shock wave emitter. The other electrode of each pair each electrode pair may be formed by a respective electrode that is positioned adjacent to one of the respective apertures of emitter bands 403 or 423.

As shown, three electrodes 405, 407, and 409 are arranged adjacent to emitter band 403. Each of the respective electrodes 405, 407, and 409 form one electrode of three electrode pairs completed by emitter band 403. For instance, electrode 405 and emitter band 403 may form a first electrode pair, electrode 407 and emitter band 403 may form a second electrode pair, and so on. Electrodes 405, 407, and 409 may be formed by uninsulated ends of respective conductive elements 414, 415, and 416. The conductive elements 414, 415, and 416 may be disposed at least partially within cavities in an outer wall of the catheter body 401 and/or at least partially within internal lumens extending along the catheter body 401. The conductive elements 414, 415, and 416 may be insulated conductive wires, and the electrodes 405, 407, and 409 may be uninsulated regions (e.g., sides or ends) of those wires. Any of electrodes 405, 407, and 409 may additionally or alternatively include a conductive material (e.g., a conductive sheath) soldered, clamped, welded, or otherwise electrically connected to a conductive portion of a respective conductive element 414, 415, and 416. Electrodes 405, 407, and 409 may be the same material as the respective conductive elements 414, 415, and 416 or may be formed at least partially of a different material from the respective conductive elements 414, 415, and 416.

Each of electrodes 405, 407, and 409 may be positioned to align with one of the respective apertures 440, 441, and 442 of emitter band 403. Electrode 405 may be positioned radially inwardly of the emitter band 403, with its distal end positioned adjacent aperture 440. Similarly, electrode 407 may be positioned radially inwardly of the emitter band 403, with its distal end positioned adjacent to aperture 441. Electrode 409 may be positioned radially inwardly of the emitter band 403, with an uninsulated end positioned adjacent aperture 442. The electrodes 405, 407, and 409 are spaced apart from the emitter band 403 by a respective spark gap 411, 412, and 413 resulting from the respective apertures, thus forming a plurality of shock wave emitters 404, 406, and 408 positioned about the circumference of catheter body 401 for generating shock waves.

A proximal end of conductive elements 414 and 415 are electrically connected to a respective supply channel such that shock wave emitters 404 and 406 can be driven independently of one another. The conductive elements 415 and 416 may extend along the length of the catheter 400 to connect to a respective supply channel (or ground terminal). A proximal end of conductive element 414 may be electrically connected to a first supply channel 450 (for instance, a supply channel of switching circuit 800 illustrated in FIG. 8), thus electrically connecting electrode 405 to supply channel 450. A proximal end of conductive element 415 may be electrically connected to a second supply channel 452, thus electrically connecting electrode 407 to supply channel 452. Conductive element 416 is not connected to a supply channel. Rather, the proximal end of conductive element 416 forms electrode 409. Conductive element 416 extends between emitter band 403 and 423, and the distal end of conductive element 416 forms electrode 427, positioned adjacent to emitter band 423, as described in additional detail below.

As shown, three electrodes 425, 427, and 429 are arranged adjacent to emitter band 423. Each of electrodes 425, 427, and 429 form one electrode of three electrode pairs completed by emitter band 423. For instance, electrode 425 and emitter band 423 may form a first electrode pair, electrode 427 and emitter band 423 may form a second electrode pair, and electrode 429 and emitter band 423 may form a third electrode pair. Electrodes 425, 427, and 429 may be formed by a respective uninsulated end of a respective conductive element 416, 417, and 418. The conductive elements 417 and 418, like conductive elements 414-416, may be insulated conductive wires (e.g., copper wires, molybdenum wires, tungsten wires, tantalum wires), and, like conductive elements 414-416, may be disposed in cavities in an outer wall of the catheter body 401 and/or within internal lumens extending along at least a portion of the catheter body. Any of electrodes 425, 427, and 429 may additionally or alternatively include a conductive material (e.g., a conductive sheath) soldered, clamped, welded, or otherwise electrically connected to a conductive portion of a respective insulated wire 416, 417, and 418 and may be the same material as the respective conductive elements or may be formed at least partially of a different material from the respective conductive elements.

Each of electrodes 425, 427, and 429 may be positioned to align with one of the respective apertures 443, 444, and 445. Electrode 425 may be positioned radially inwardly of the emitter band 423, with an uninsulated end positioned adjacent aperture 443. Similarly, electrode 427 may be positioned radially inwardly of the emitter band 423, with an uninsulated end positioned adjacent to aperture 444, and electrode 429 may be positioned radially inwardly of the emitter band 423, with an uninsulated end positioned adjacent aperture 445. The electrodes 425, 427, and 429 are spaced apart from the emitter band 423 by a respective spark gap 431, 432, and 433 resulting from the respective apertures, thus forming a plurality of shock wave emitters 424, 426, and 428 positioned about the circumference of catheter body 401 for generating shock waves.

A proximal end of conductive element 417 may be electrically connected to a third supply channel 454, thus electrically connecting electrode 425 to supply channel 454. A proximal end of conductive element 418 may be electrically connected to a ground terminal 456, thus electrically connecting electrode 429 to ground terminal 456. Conductive element 418 may extend along the length of catheter 400 to connect to the ground terminal 456. As described above, rather than electrically connecting conductive element 416 to either a supply channel or a ground terminal, it may instead extend between conductive emitter band 403 and 423, electrically connecting the two emitter bands 403 and 423 via electrode 427 positioned adjacent to emitter band 423 and electrode 409 positioned adjacent to emitter band 403. Conductive element 416 may be an insulated wire that extends between emitter bands 403 and 423 with two uninsulated ends (electrodes 409 and 427) respectively positioned adjacent to each of the emitter bands such that each of the uninsulated ends are aligned with a respective aperture 442 and 444, as shown.

Accordingly, when either of supply channels 450 or 452 are pulsed, at least four shock waves may be generated (two at emitter band 403 and two at emitter band 423). However, when supply channel 424 is pulsed, only two shock wave may be generated (each at emitter band 423) because conductive element 418 and corresponding electrode 429 positioned adjacent to emitter band 423 are connected to the ground terminal 456. Accordingly, the current will follow the path of least resistance to ground via conductive element 418 rather than to emitter band 403 via conductive element 416.

For instance, when the first supply channel 450 electrically connected to electrode 405 via conductive element 414 is pulsed (e.g., when a voltage differential is applied across conductive elements 414 and 418), a shock wave may simultaneously be generated at shock wave emitters 404, 408, 426, and 428, which are electrically connected in series. As used herein, simultaneous generation may refer to the nearly/approximately simultaneous, or simultaneous, shock wave generation at a plurality of shock wave emitters connected in series on the same power supply channel. A shock wave may be generated at shock wave emitter 404 due to the spark caused by a voltage differential between electrode 405 and emitter band 403. Because the current will follow the path of least resistance to the ground terminal 456, which is electrically connected to shock wave emitter 428 at emitter band 423, the current will flow between the emitter band 403 and electrode 409 at shock wave emitter 408, which is electrically connected to emitter band 423 via conductive element 416, rather than between the emitter band 403 and electrode 407, which in this example is connected to a second positive terminal 452. Accordingly, a second shock wave may be generated at shock wave emitter 408 due to the spark caused by the voltage differential across the spark gap 413 between electrode 409 and emitter band 403. Because electrode 409 is electrically connected to shock wave emitter 426 positioned at the second emitter band 423 via insulated wire 416, a third shock wave may be generated at shock wave emitter 426 due to the spark caused by a voltage differential across spark gap 432 between electrode 427 and emitter band 423. Finally, a fourth shock wave may be generated at shock wave emitter 428 due to the spark caused by a voltage differential across the spark gap 433 between electrode 429 and emitter band 423 as the current follows the path of resistance to ground terminal 456 via insulated wire 418 (e.g., due to a voltage differential between wire 414 connected to a positive supply channel and wire 418 connected to ground or a negative terminal).

If the pulse is instead applied to the supply channel 452 connected to shock wave emitter 406 via insulated wire 415 (e.g., across wire 415 connected to the positive supply channel 452 and wire 418 connected to ground/negative terminal 456), emitters 406, 408, 426, and 428 may simultaneously generate shock waves in the same manner as when the pulse is applied to shock wave emitter 404 via supply channel 450, and no shock wave will be generated at shock wave emitter 404.

In contrast to the two scenarios above, when a pulse is applied to shock wave emitter 424 via the third supply channel 454 (e.g., when a voltage differential is applied across insulated wire 417 connected to a positive supply channel 454 and insulated wire 418 connected to ground terminal/negative terminal 456), only two shock waves may be generated, both at the more distal emitter band 423. A first shock wave may be generated at shock wave emitter 424 due to the spark caused by the voltage differential across spark gap 431 between electrode 425 and emitter band 423. Simultaneously, a second shock wave may be emitted at shock wave emitter 428 due to the spark caused by the voltage differential across spark gap 433 between electrode 429 and emitter band 423 as the current follows the path of least resistance to ground and/or the negative terminal 456 connected to emitter 428 via conductive element 418. One having skill in the art would understand that any of the supply channels may serve as the ground terminal and the ground terminal could serve as a replacement supply channel, which may alter the flow of current through the conductive elements of the catheter. That is, changes to the electrical configuration of the catheter are possible without deviating from the scope of the disclosure.

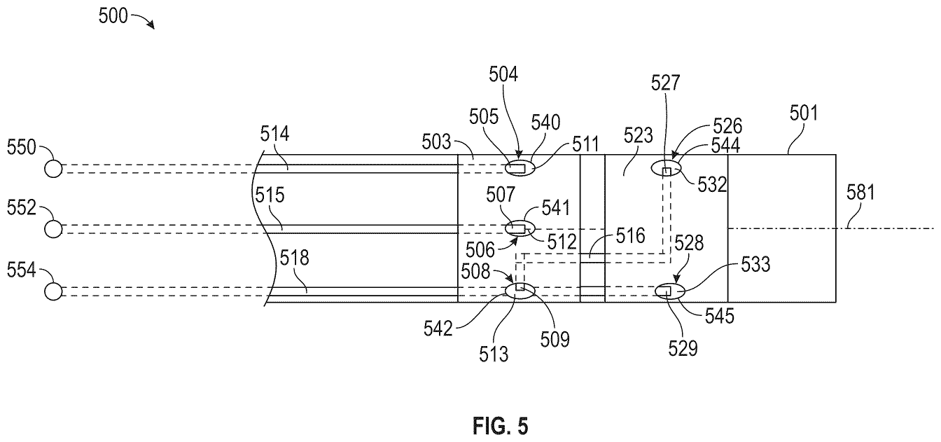

FIG. 5 illustrates an example that includes at least two shock wave emitter bands, each forming multiple shock wave emitters (the view of FIG. 5 depicts the emitter band as if it were cut and laid out flat). Catheter 500 includes many of the elements described above with respect to catheter 400 of FIG. 4 but includes only two shock wave emitters at the second emitter band 523, and thus may require only two supply channels, respectively connected to insulated wires 514 and 515. The conductive elements 514 and 515 may extend along the length of the catheter 400 to connect to a respective supply channel. Accordingly, shock waves may be generated using catheter 500 by applying one or more suitably high voltage pulses to (e.g., applying a voltage differential) across wire 514 connected to a first supply channel and wire 518 connected to ground/a negative terminal and/or by applying a voltage differential across wire 515 connected to a second supply channel and wire 518. Supplying one or more suitably high voltage pulses to either the first supply channel or the second supply channel may result in four shock waves in a similar manner to that described above with reference to the supply channels respectively connected to conductive elements 414 and 415. Catheter 500 is described in additional detail below.

The catheter 500 of FIG. 5 includes a catheter body 401 and two conductive emitter bands 503 and 523 positioned at two different locations (emitter band 523 positioned distally of emitter band 503) on the catheter body 501. Each of emitter bands 503 and 523 form a portion of multiple shock wave emitters that may be positioned on catheter 500 for generating shock waves. Emitter bands 503 and 523 may be conductive cylindrical bands extending around the catheter body 501. Emitter band 503 and 523 each include a respective set of apertures. Emitter band 503 includes apertures 540, 541, and 542. Emitter band 523 includes apertures 544 and 445. The apertures on emitter band 503 are spaced from one another in the circumferential direction about longitudinal axis 581 of catheter body 501. Similarly, the apertures on emitter band 523 are spaced from one another in the circumferential direction about longitudinal axis 581 of catheter body 501.

The three apertures on emitter band 503 may be equally spaced apart 120 degrees from one another about longitudinal axis 581 and may extend through a thickness of the respective emitter band 503. The two apertures on emitter band 523 may be spaced apart 180 degrees from one another about longitudinal axis 581 and may extend through a thickness of the respective emitter band 523. Emitter bands 503 and 523 respectively form one electrode of each electrode pair that corresponds to a respective shock wave emitter. The other electrode of each pair each electrode pair may be formed by a respective electrode that is positioned adjacent to one of the respective apertures of emitter bands 503 or 523.

As shown, three electrodes 505, 507, and 509 are arranged adjacent to emitter band 503. Each of the respective electrodes 505, 507, and 509 form one electrode of three electrode pairs completed by emitter band 503. For instance, electrode 505 and emitter band 503 may form a first electrode pair, electrode 507 and emitter band 503 may form a second electrode pair, and so on. Electrodes 505, 507, and 509 may be formed by uninsulated ends of respective conductive elements 514, 515, and 516. The conductive elements 514, 515, and 516 may be disposed at least partially within cavities in an outer wall of the catheter body 501 and/or at least partially within internal lumens extending along the catheter body 501. The conductive elements 514, 515, and 516 may be insulated conductive wires (e.g., copper wires, molybdenum wires, tungsten wires, tantalum wires), and the electrodes may 505, 507, and 509 may be uninsulated ends of those wires. Any of electrodes 505, 507, and 509 may additionally or alternatively include a conductive material (e.g., a conductive sheath) soldered, clamped, welded, or otherwise electrically connected to a conductive portion of a respective conductive element 514, 515, and 516. Electrodes 505, 507, and 509 may be the same material as the respective conductive elements 514, 515, and 516 or may be formed at least partially of a different material from the respective conductive elements 514, 515, and 516.

Each of electrodes 505, 507, and 509 may be positioned to align with one of the respective apertures 540, 541, and 542. Electrode 505 may be positioned radially inwardly of the emitter band 503, with its distal end positioned adjacent aperture 540. Similarly, electrode 507 may be positioned radially inwardly of the emitter band 503, with its distal end positioned adjacent to aperture 541. Electrode 509 may be positioned radially inwardly of the emitter band 503, with an uninsulated end positioned adjacent aperture 542. The electrodes 505, 507, and 509 are spaced apart from the emitter band 503 by a respective spark gap 511, 512, and 513 resulting from the respective apertures, thus forming a plurality of shock wave emitters 504, 506, and 508 positioned about the circumference of catheter body 401 for generating shock waves.

A proximal end of conductive elements 514 and 515 are electrically connected to a respective supply channel such that shock wave emitters 504 and 506 can be driven independently of one another. A proximal end of conductive element 514 may be electrically connected to a first supply channel 550 (for instance, a supply channel of switching circuit 800 illustrated in FIG. 8), thus electrically connecting electrode 505 to supply channel 550. A proximal end of conductive element 515 may be electrically connected to a second supply channel 552, thus electrically connecting electrode 507 to supply channel 552. Conductive element 516 is not connected to a supply channel. Rather, the proximal end of conductive element 516 forms electrode 509. Conductive element 516 extends between emitter band 503 and 523, and the distal end of conductive element 516 forms electrode 527, positioned adjacent to emitter band 523, as described in additional detail below.

As shown, two electrodes 527 and 529 are arranged adjacent to emitter band 523. Each of electrodes 527 and 529 form one electrode of two electrode pairs completed by emitter band 523. For instance, electrode 527 and emitter band 523 may form a first electrode pair and electrode 529 and emitter band 523 may form a second electrode pair. Electrodes 527 and 529 may be formed by a respective uninsulated end of a respective conductive element 516 and 518. The conductive element 518, like conductive elements 514-516, may be an insulated conductive wire (e.g., copper wire, molybdenum wire, tungsten wire, tantalum wire), and, like conductive elements 514-516, may be disposed in cavities in an outer wall of the catheter body 501 and/or within internal lumens extending along at least a portion of the catheter body 501. Any of electrodes 527 and 529 may additionally or alternatively include a conductive material (e.g., a conductive sheath) soldered, clamped, welded, or otherwise electrically connected to a conductive portion of a respective conductive element 516 and 518 and may be the same material as the respective conductive elements or may be formed at least partially of a different material from the respective conductive elements.

Each of electrodes 527 and 529 may be positioned to align with one of the respective apertures 544 and 545 of emitter band 523. Electrode 527 may be positioned radially inwardly of the emitter band 523, with an uninsulated end positioned adjacent aperture 544. Similarly, electrode 529 may be positioned radially inwardly of the emitter band 523, with an uninsulated end positioned adjacent to aperture 545. The electrodes 527 and 529 are spaced apart from the emitter band 523 by a respective spark gap 532 and 533 resulting from the respective apertures, thus forming a plurality of shock wave emitters 526 and 528 positioned about the circumference of catheter body 501 for generating shock waves.

A proximal end of conductive element 518 may be electrically connected to a ground terminal 554, thus electrically connecting electrode 529 to ground terminal 554. Conductive element 518 may extend along the length of catheter 500 to connect to ground terminal 554. As described above, rather than electrically connecting conductive element 516 to either a supply channel or a ground terminal, it may instead extend between conductive emitter band 503 and 523, electrically connecting the two bands via electrode 527 positioned adjacent to emitter band 523 and electrode 509 positioned adjacent to emitter band 503. Conductive element 516 may be an insulated wire that extends between emitter bands 503 and 523 with two uninsulated ends (electrodes 509 and 527) respectively positioned adjacent to each of the emitter bands such that each of the uninsulated ends are aligned with a respective aperture 542 and 544, as shown.

Accordingly, when either of supply channels 550 or 552 are pulsed, at least four shock waves may be generated (two at emitter band 503 and two at emitter band 523). For instance, when the first supply channel 550 electrically connected to electrode 505 via conductive element 514 is pulsed (e.g., when a voltage differential is applied across conductive elements 514 and 518), a shock wave may simultaneously be generated at shock wave emitters 504, 508, 526, and 528. A shock wave may be generated at shock wave emitter 504 due to the spark caused by a voltage differential between electrode 505 and emitter band 503. Because the current will follow the path of least resistance to the ground terminal 554, which is electrically connected to shock wave emitter 528 at emitter band 523, the current will flow between the emitter band 503 and electrode 509 at shock wave emitter 508, which is electrically connected to emitter band 523 via conductive element 516, rather than between the emitter band 503 and electrode 507, which in this example is connected to a second positive terminal 552. Accordingly, a second shock wave may be generated at shock wave emitter 508 due to the spark caused by the voltage differential across the spark gap 513 between electrode 509 and emitter band 503. Because electrode 509 is electrically connected to shock wave emitter 526 positioned at the second emitter band 523 via insulated wire 516, a third shock wave may be generated at shock wave emitter 526 due to the spark caused by a voltage differential across spark gap 532 between electrode 527 and emitter band 523. Finally, a fourth shock wave may be generated at shock wave emitter 528 due to the spark caused by a voltage differential across the spark gap 533 between electrode 529 and emitter band 523 as the current follows the path of resistance to ground terminal 554 via insulated wire 418 (e.g., due to a voltage differential between wire 514 connected to a positive supply channel and wire 518 connected to ground or a negative terminal).

If the pulse is instead applied to the supply channel 552 connected to shock wave emitter 506 via conductive element 515 (e.g., across wire 515 connected to the positive supply channel 552 and wire 518 connected to ground/negative terminal 554), emitters 506, 508, 526, and 528 may simultaneously generate shock waves in the same manner as when the pulse is applied to shock wave emitter 504 via supply channel 550, and no shock wave will be generated at shock wave emitter 504. One having skill in the art would understand that any of the supply channels (550 and 552) may serve as the ground terminal and ground terminal 554 could serve as a replacement supply channel, which may alter the flow of current through the conductive elements of the catheter. That is, changes to the electrical configuration of the catheter are possible without deviating from the scope of the disclosure.

FIG. 6 illustrates an example catheter 600 that includes at least two emitter bands (the view of FIG. 6 depicts the emitter band as if it were cut and laid out flat). Catheter 600 is similar to catheter 500 depicted in FIG. 5 in that it includes two emitter bands 603 and 623, but catheter 600 does not include a conductive element that conducts current from one emitter band to the other (e.g., as conductive element 516 of catheter 500 does). Rather, emitter bands 603 and 623 are commonly connected to wire 618, which is connected to a ground terminal (or negative terminal) 658. The current may flow from the emitter bands 603 and 623 to wire 618, rather than from wire 618 to the emitter bands. Because of this, the current may not transfer from band 602 to band 623, or vice versa.

As shown, catheter 600 includes a catheter body 601 and two conductive emitter bands 603 and 623 positioned at two different locations (emitter band 623 positioned distally of emitter band 603) on the catheter body 601. Each of emitter bands 603 and 623 form a portion of multiple shock wave emitters that may be positioned on catheter 600 for generating shock waves. Emitter bands 603 and 623 may be conductive cylindrical bands extending around the catheter body 601. Emitter band 603 and 623 each include a respective set of apertures. Emitter band 603 includes apertures 640, 641, and 642. Emitter band 623 includes apertures 643, 644, and 645. The apertures on emitter band 603 are spaced from one another in the circumferential direction about longitudinal axis 681 of catheter body 601. Similarly, the apertures on emitter band 623 are spaced from one another in the circumferential direction about longitudinal axis 681 of catheter body 601. The apertures on each of emitter bands 603 and 623 may be equally spaced apart 120 degrees from one another about longitudinal axis 681 and may extend through a thickness of the respective emitter bands 603 and 623. Emitter bands 603 and 623 respectively form one electrode of each electrode pair that corresponds to a respective shock wave emitter. The other electrode of each pair each electrode pair may be formed by a respective electrode that is positioned adjacent to one of the respective apertures of emitter bands 603 or 623.

As shown, three electrodes 605, 607, and 609 are arranged adjacent to emitter band 603. Each of the respective electrodes 605, 607, and 609 form one electrode of three electrode pairs completed by emitter band 603. For instance, electrode 605 and emitter band 603 may form a first electrode pair, electrode 607 and emitter band 603 may form a second electrode pair, and so on. Electrodes 605, 607, and 609 may be formed by uninsulated portions of respective conductive elements 614, 615, and 618. The conductive elements 614, 615, and 618 may be disposed at least partially within cavities in an outer wall of the catheter body 601 and/or at least partially within internal lumens extending along the catheter body 601. The conductive elements 614, 615, and 618 may be insulated conductive wires (e.g., copper wires, molybdenum wires, tungsten wires, tantalum wires), and the electrodes may 605, 607, and 609 may be uninsulated portions (e.g., uninsulated ends or other uninsulated conductive portions) of those wires. Any of electrodes 605, 607, and 609 may additionally or alternatively include a conductive material (e.g., a conductive sheath) soldered, clamped, welded, or otherwise electrically connected to a conductive portion of a respective conductive element 614, 615, and 618. Electrodes 605, 607, and 609 may be the same material as the respective conductive elements 614, 615, and 618 or may be formed at least partially of a different material from the respective conductive elements 614, 615, and 618.

Each of electrodes 605, 607, and 609 may be positioned to align with one of the respective apertures 640, 641, and 642. Electrode 605 may be positioned radially inwardly of the emitter band 603, with its distal end positioned adjacent aperture 640. Similarly, electrode 607 may be positioned radially inwardly of the emitter band 603, with its distal end positioned adjacent to aperture 641. Electrode 609 may be positioned radially inwardly of the emitter band 603, with an uninsulated portion positioned adjacent aperture 642. The electrodes 605, 607, and 609 are spaced apart from the emitter band 603 by a respective spark gap 611, 612, and 613 resulting from the respective apertures, thus forming a plurality of shock wave emitters 604, 606, and 608 positioned about the circumference of catheter body 601 for generating shock waves.

A proximal end of conductive elements 614 and 615 are electrically connected to a respective supply channel such that shock wave emitters 604 and 606 can be driven independently of one another. A proximal end of conductive element 614 may be electrically connected to a first supply channel 650 (for instance, a supply channel of switching circuit 800 illustrated in FIG. 8), thus electrically connecting electrode 605 to supply channel 650. A proximal end of conductive element 615 may be electrically connected to a second supply channel 654, thus electrically connecting electrode 607 to supply channel 654. Conductive element 618 is electrically connected to ground terminal 658, thus electrically connecting electrode 609 to ground.

As shown, three electrodes 625, 627, and 629 are arranged adjacent to emitter band 623. Each of electrodes 625, 627, and 629 form one electrode of three electrode pairs completed by emitter band 623. For instance, electrode 625 and emitter band 623 may form a first electrode pair, electrode 627 and emitter band 623 may form a second electrode pair, and electrode 629 and emitter band 623 may form a third electrode pair. Electrodes 625, 627, and 629 may be formed by a respective uninsulated end of a respective conductive element 616, 617, and 618. The conductive elements 616 and 617 may be insulated conductive wires (e.g., copper wires, molybdenum wires, tungsten wires, tantalum wires), and, like conductive elements 614, 615, and 618, may be disposed in cavities in an outer wall of the catheter body 601 and/or within internal lumens extending along at least a portion of the catheter body. Any of electrodes 625, 627, and 629 may additionally or alternatively include a conductive material (e.g., a conductive sheath) soldered, clamped, welded, or otherwise electrically connected to a conductive portion of a respective insulated wire 616, 617, and 618 and may be the same material as the respective conductive elements or may be formed at least partially of a different material from the respective conductive elements.

Each of electrodes 625, 627, and 629 may be positioned to align with one of the respective apertures 643, 644, and 645. Electrode 625 may be positioned radially inwardly of the emitter band 623, with an uninsulated end positioned adjacent aperture 643. Similarly, electrode 627 may be positioned radially inwardly of the emitter band 623, with an uninsulated end positioned adjacent to aperture 644, and electrode 629 may be positioned radially inwardly of the emitter band 623, with an uninsulated end positioned adjacent aperture 645. The electrodes 625, 627, and 629 are spaced apart from the emitter band 623 by a respective spark gap 631, 632, and 633 resulting from the respective apertures, thus forming a plurality of shock wave emitters 624, 626, and 628 positioned about the circumference of catheter body 601 for generating shock waves.

A proximal end of conductive element 616 may be electrically connected to a third supply channel 652, thus electrically connecting electrode 625 to supply channel 652. A proximal end of conductive element 617 may be electrically connected to a fourth supply channel 656, thus electrically connecting electrode 627 to supply channel 656. As described above a proximal end of conductive element 618 may be connected to ground terminal 658, thus electrically connecting electrode 629 to ground. Conductive elements 614, 615, 616, 617, and 618 may extend along the length of catheter 600 to connect to a respective supply channel or ground terminal. The supply channels and/or ground terminal may be provided on a switching circuit (e.g., switching circuit 800).

Supplying one or more suitably high voltage pulses to any one of the supply channels 650, 652, 654, and 656 may generate at least two shock waves. A first shock wave may be generated at the shock wave emitter electrically connected to the pulsed channel and a second shock wave may be generated at one of two shock wave emitters that are electrically connected to ground terminal 658. For example, if channel 650 is pulsed, then a shock wave may be generated at shock wave emitter 604 and shock wave emitter 608, due to the voltage differential applied across conductive element 614 connected to supply channel 650 and conductive element 618 connected to ground terminal 658. If channel 654 is pulsed, then a shock wave may be generated at shock wave emitter 606 and shock wave emitter 608, due to the voltage differential applied across conductive element 615 connected to supply channel 654 and conductive element 618 connected to ground terminal 658. If channel 652 is pulsed, then a shock wave may be generated at shock wave emitter 624 and shock wave emitter 628, due to the voltage differential applied across conductive element 616 connected to channel 652 and conductive element 618 connected to ground terminal 658. Finally, if supply channel 656 is pulsed, then a shock wave may be generated at shock wave emitter 626 and shock wave emitter 628, due to the voltage differential applied across conductive element 617 connected to channel 656 and conductive element 618 connected to ground terminal 658.

One having skill in the art would understand that any of the supply channels may serve as the ground terminal and the ground terminal could serve as a replacement supply channel, which may alter the flow of current through the conductive elements of the catheter. That is, changes to the electrical configuration of the catheter are possible without deviating from the scope of the disclosure.

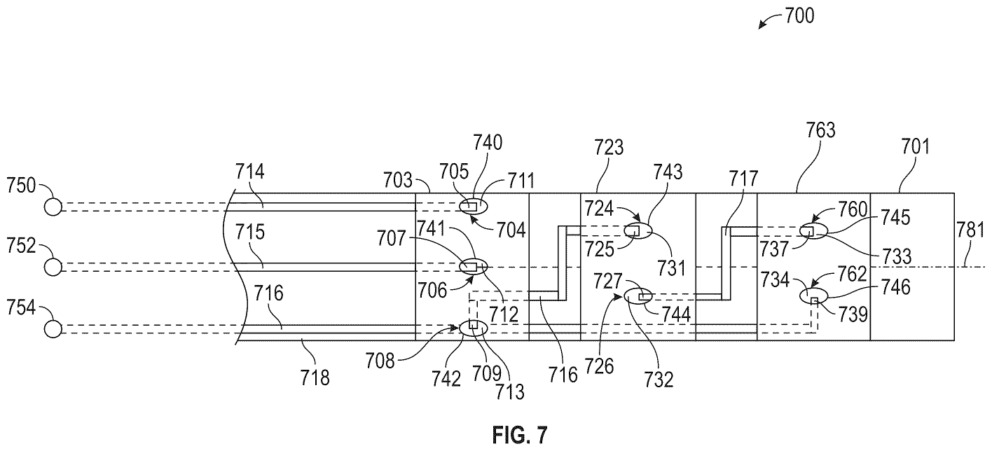

FIG. 7 illustrates an example of a catheter 700 that includes at least three emitter bands (the view of FIG. 7 depicts the emitter band as if it were cut and laid out flat). Catheter 700 includes a catheter body 701 and three emitter bands 703, 723, and 763, positioned at three different locations long the catheter body 701 electrically connected by respective conductive elements that extend between the emitter bands (similar to conductive elements 416 of catheter 400), as described in additional detail below.

Each of emitter bands 703, 723, and 763 form a portion of multiple shock wave emitters that may be positioned on catheter 700 for generating shock waves. Emitter bands 703, 723, and 763 may be conductive cylindrical bands extending around at least a portion of the catheter body 701. Emitter bands 703, 723, and 763 each include a respective set of apertures. Emitter band 703 includes apertures 740, 741, and 742. Emitter band 723 includes apertures 743, and 744. Emitter band 763 includes apertures 745 and 746. The apertures on emitter band 703 are spaced from one another in the circumferential direction about longitudinal axis 781 of catheter body 701. Similarly, the apertures on emitter band 723 are spaced from one another in the circumferential direction about longitudinal axis 781 of catheter body 701, and the apertures on emitter band 763 are spaced from one another in the circumferential direction about longitudinal axis 781 of catheter body 701. The apertures on emitter band 703 may be spaced apart 120 degrees from one another, and the apertures on both of emitter bands 723 and 763 may spaced apart 180 degrees from one another about longitudinal axis 781. The apertures on each of the emitter bands may extend through a thickness of the respective emitter bands 703, 723, and 763. Emitter bands 703, 723, and 763 respectively form one electrode of each electrode pair that corresponds to a respective shock wave emitter. The other electrode of each pair each electrode pair may be formed by a respective electrode that is positioned adjacent to one of the respective apertures of emitter bands 703, 723, and 763.

As shown, three electrodes 705, 707, and 709 are arranged adjacent to emitter band 703. Each of the respective electrodes 705, 707, and 709 form one electrode of three electrode pairs completed by emitter band 703. For instance, electrode 705 and emitter band 703 may form a first electrode pair, electrode 707 and emitter band 703 may form a second electrode pair, and so on. Electrodes 705, 707, and 709 may be formed by uninsulated ends of respective conductive elements 714, 715, and 716. The conductive elements 714, 715, and 716 may be disposed at least partially within cavities in an outer wall of the catheter body 701 and/or at least partially within internal lumens extending along the catheter body 701. The conductive elements 714, 715, and 716 may be insulated conductive wires (e.g., copper wire, molybdenum wire, tungsten wire, tantalum wire), and the electrodes may 705, 707, and 709 may be uninsulated ends of those wires. Any of electrodes 705, 707, and 709 may additionally or alternatively include a conductive material (e.g., a conductive sheath) soldered, clamped, welded, or otherwise electrically connected to a conductive portion of a respective conductive element 714, 715, and 716. Electrodes 705, 707, and 709 may be the same material as the respective conductive elements 714, 715, and 716 or may be formed at least partially of a different material from the respective conductive elements 714, 715, and 716.

Each of electrodes 705, 707, and 709 may be positioned to align with one of the respective apertures 740, 741, and 742. Electrode 705 may be positioned radially inwardly of the emitter band 703, with its distal end positioned adjacent aperture 740. Similarly, electrode 707 may be positioned radially inwardly of the emitter band 703, with its distal end positioned adjacent to aperture 741. Electrode 709 may be positioned radially inwardly of the emitter band 703, with an uninsulated end positioned adjacent aperture 742. The electrodes 705, 707, and 709 are spaced apart from the emitter band 703 by a respective spark gap 711, 712, and 713 resulting from the respective apertures, thus forming a plurality of shock wave emitters 704, 706, and 708 positioned about the circumference of catheter body 401 for generating shock waves.

A proximal end of conductive elements 714 and 715 are electrically connected to a respective supply channel such that shock wave emitters 704 and 706 can be driven independently of one another. A proximal end of conductive element 714 may be electrically connected to a first supply channel 750 (for instance, a supply channel of switching circuit 800 illustrated in FIG. 8), thus electrically connecting electrode 705 to supply channel 750. A proximal end of conductive element 715 may be electrically connected to a second supply channel 752, thus electrically connecting electrode 707 to supply channel 752. Conductive elements 714 and 715 may extend along the length of catheter 700 to connect to supply channels 750 and 752, respectively. The supply channels 750 and 752 may be included on a switching circuit (e.g., switching circuit 800). Conductive element 716 is not connected to a supply channel. Rather, the proximal end of conductive element 716 forms electrode 709. Conductive element 716 extends between emitter band 703 and 723, and the distal end of conductive element 716 forms electrode 727, positioned adjacent to emitter band 723, as described in additional detail below.

As shown, two electrodes 725 and 727 are arranged adjacent to emitter band 723. Each of electrodes 725 and 727 form one electrode of two electrode pairs completed by emitter band 723. For instance, electrode 725 and emitter band 723 may form a first electrode pair, and electrode 727 and emitter band 723 may form a second electrode pair. Electrodes 725 and 727 may be formed by a respective uninsulated end of a respective conductive element 716 and 717. Like conductive element 716, conductive element 717 may be an insulated conductive wire (e.g., copper wire, molybdenum wire, tungsten wire, tantalum wire), and, like conductive elements 714, 715, and 716, may be disposed in a cavity in an outer wall of the catheter body 701 and/or within internal lumens extending along at least a portion of the catheter body. Any of electrodes 725 and 727 may additionally or alternatively include a conductive material (e.g., a conductive sheath) soldered, clamped, welded, or otherwise electrically connected to a conductive portion of a respective insulated wire 716 and 717 and may be the same material as the respective conductive elements or may be formed at least partially of a different material from the respective conductive elements.

Each of electrodes 725 and 727 may be positioned to align with one of the respective apertures 743 and 744. Electrode 725 may be positioned radially inwardly of the emitter band 723, with an uninsulated end positioned adjacent aperture 743. Similarly, electrode 727 may be positioned radially inwardly of the emitter band 723, with an uninsulated end positioned adjacent to aperture 744. The electrodes 725 and 727 are spaced apart from the emitter band 723 by a respective spark gap 731 and 732 resulting from the respective apertures, thus forming a plurality of shock wave emitters 724 and 726 positioned about the circumference of catheter body 701 for generating shock waves. As described above, a proximal end of conductive element 716 forms electrode 709 positioned adjacent to emitter band 703. Conductive element 717 extends between emitter band 723 and 763, and a distal end of conductive element 717 forms an electrode 737 positioned adjacent to emitter band 763, as described further below.

As shown, two electrodes 737 and 739 are arranged adjacent to emitter band 763. Each of electrodes 737 and 739 form one electrode of two electrode pairs completed by emitter band 763. For instance, electrode 737 and emitter band 763 may form a first electrode pair, and electrode 739 and emitter band 763 may form a second electrode pair. Electrodes 737 and 739 may be formed by a respective uninsulated end of a respective conductive element 717 and 718. Like conductive element 717, conductive element 718 may be an insulated conductive wire (e.g., copper wire, molybdenum wire, tungsten wire, tantalum wire). Conductive element 718 may be disposed in a cavity in an outer wall of the catheter body 701 and/or within internal lumens extending along at least a portion of the catheter body. Any of electrodes 737 and 739 may additionally or alternatively include a conductive material (e.g., a conductive sheath) soldered, clamped, welded, or otherwise electrically connected to a conductive portion of a respective insulated wire 717 and/or 718 and may be the same material as the respective conductive elements or may be formed at least partially of a different material from the respective conductive elements.

Each of electrodes 737 and 739 may be positioned to align with one of the respective apertures 745 and 746. Electrode 737 may be positioned radially inwardly of the emitter band 763, with an uninsulated end positioned adjacent aperture 745. Similarly, electrode 739 may be positioned radially inwardly of the emitter band 763, with an uninsulated end positioned adjacent to aperture 746. The electrodes 737 and 739 are spaced apart from the emitter band 763 by a respective spark gap 733 and 734 resulting from the respective apertures, thus forming a plurality of shock wave emitters 760 and 762 positioned about the circumference of catheter body 701 for generating shock waves. As described above, a proximal end of conductive element 717 forms electrode 727 positioned adjacent to emitter band 723. Conductive element 718 extends along the length of the catheter body 701 to electrically connect to ground terminal 754, thus electrically connecting electrode 739 to ground. The ground terminal 754 may be included on a switching circuit (e.g., switching circuit 800).

By supplying one or more suitably high voltage pulses to either of the supply channels 750 or 752, at least two shock waves may be generated by two shock wave emitters positioned at each of the three emitter bands, as described in further detail below.

For instance, when either of the two supply channels 750 or 752 are pulsed (e.g., when a voltage differential is applied across conductive element 714 and conductive element 718 via supply channel 750, or when a voltage differential is applied across conductive element 715 and conductive element 718 via supply channel 752), at least two shock waves may be simultaneously generated by two respective shock wave emitters positioned at each of emitter bands 703, 723, and 763.

When the first supply channel 750 electrically connected to electrode 705 is pulsed, a shock wave may simultaneously be generated at shock wave emitters 704 and 708 at emitter band 703, at shock wave emitters 724 and 726 at emitter band 723, and at shock wave emitters 760 and 762 at emitter band 763. A shock wave may be generated at shock wave emitter 704 due to the spark caused by a voltage differential across spark gap 711 between electrode 705 and emitter band 703. Because the current will follow the path of least resistance to the ground terminal, which is connected to shock wave emitter 762 at emitter band 763, the current will flow across the spark gap 713 between the emitter band 703 and electrode 709 at shock wave emitter 708, which is electrically connected to emitter band 723 (e.g., the next band in the series), rather than across the spark gap 712 between the emitter band 703 and electrode 707, which in this example is connected to a second supply channel 752. Accordingly, a second shock wave may be generated at shock wave emitter 708 due to the spark caused by the voltage differential across spark gap 713 between electrode 709 and emitter band 703.

Because electrode 709 is electrically connected to shock wave emitter 724 positioned at the second emitter band 723 via insulated wire 716, a third shock wave may be generated at shock wave emitter 724 due to the spark caused by a voltage differential across spark gap 731 between electrode 725 and emitter band 723. A fourth shock wave may be generated at shock wave emitter 726 due to the spark caused by a voltage differential across spark gap 733 between electrode 729 and emitter band 723 as the current follows the path of resistance to the ground terminal 754. A fifth shock wave may be generated at shock wave emitter 760 at emitter band 763 due to the spark caused by a voltage differential across spark gap 733 between electrode 737 and emitter band 763. Finally, a sixth shock wave may be generated at shock wave emitter 762 due to the spark caused by a voltage differential across spark gap 734 between electrode 739 and emitter band 763 as the current follows wire 718 to ground terminal (or negative terminal) 754.

If the pulse is instead applied to the supply channel 752 connected to shock wave emitter 706 via insulated wire 715 (e.g., across wire 715 connected to the supply channel 752 and wire 718 connected to ground and/or a negative terminal 754), emitters 706, 708, 724, 726, 760, and 762 may simultaneously generate shock waves in the same manner as when the pulse is applied to shock wave emitter 704, and no shock wave will be generated at shock wave emitter 704. One having skill in the art would understand that any of the supply channels may serve as the ground terminal and the ground terminal could serve as a replacement supply channel, which may alter the flow of current through the conductive elements of the catheter. That is, changes to the electrical configuration of the catheter are possible without deviating from the scope of the disclosure.

As described throughout, one or more shock wave emitters provided on any of the catheters described herein may be electrically connected to a voltage source via a supply channel of a switching circuit, and one or more shock wave emitters may be connected to a ground terminal of the switching circuit. FIG. 8 illustrates an exemplary switching circuit 800 and corresponding wiring schematic that may correspond to any of the catheters. As illustrated, the switching circuit may include 1, 2, . . . . N supply channels to which 1, 2, . . . . N corresponding shock wave emitters may be respectively electrically connected via a supply wire. Additionally, one or more shock wave emitters may connected to a ground terminal by an insulated wire. As illustrated, supplying one or more suitably high voltage pulses to any one of the supply channels will generate a shock wave at least at the respective shock wave emitter electrically connected to the supply channel (e.g., via an insulated wire) as well as at a shock wave emitter connected to the ground terminal via another insulated wire. In some examples, additional shock wave emitters may be connected to each of the respective supply channels, as described throughout, thus resulting in more than two shock waves being generated when the respective supply channel is pulsed.

As described above, the exemplary catheters described herein may be connected to a voltage source and fluid source. The voltage source may be connected to one or more of the wires used to generate shock waves at the plurality of shock wave emitters of the catheters described herein, and the fluid source may be used to fill portions of the catheter, such as enclosure 18 described with reference to FIG. 1, with a conductive fluid, as described above.

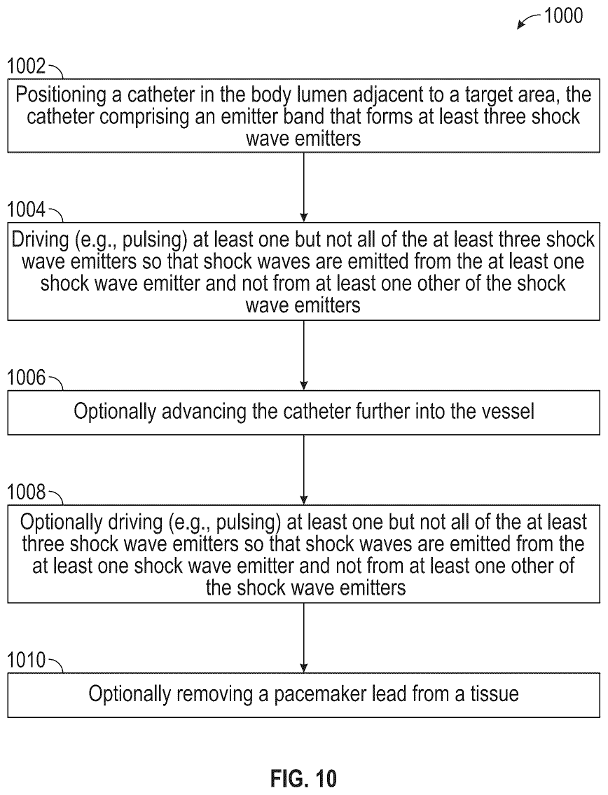

FIG. 9 illustrates an exemplary system 900 comprising a catheter 920 connected to a shock wave energy generator 930. Catheter 920 may include any combination of the features described above with reference to FIGS. 1-8 above, and/or FIGS. 10-14 below. Accordingly, catheter 920 may include a plurality of shock wave emitters 906-908. The plurality of shock wave emitters 906-908 may include laser emitters, and a plurality of optical fibers extend along the length of the catheter 920 from the emitters 906-908 to the shock wave energy generator 930. Catheter 920 may be connected to one or both of the shock wave energy generator 930 and/or fluid source 940 at proximal end 922 of the catheter 920.

Shock wave energy generator 930 may be a portable and/or rechargeable voltage source. Shock wave energy generator 930 may be a laser pulse generator. Shock wave energy generator 830 may configured to deliver high voltage pulses to a shock wave emitter of a plurality of shock wave emitters of catheter 820, wherein the high voltage pulses are between 3 kV and 20 kV, including 3 kV and 20 kV. The high voltage pulses may be between 10 kV and 20 kV, including 10 kV and 20 kV, between 15 kV and 30 kV, including 15 kV and 30 kV, and/or greater than 30 kV. The high voltage pulses may be at least 1 kV, at least 2 kV, at least 3 kV, at least 4 kV, at least 5 kV, at least 6 kV, at least 7 kV, at least 8 kV, at least 9 kV, at least 10 kV, at least 11 kV, at least 12 kV, at least 13 kV, at least 14 kV, at least 15 kV, at least 16 kV, at least 17 kV, at least 18 kV, at least 19 kV, and/or at least 20 kV. The high voltage pulses may be no more than 20 kV, no more than 19 kV, no more than 18 kV, no more than 17 kV, no more than 16 kV, no more than 15 kV, no more than 14 kV, no more than 13 kV, no more than 12 kV, no more than 11 kV, no more than 10 kV, no more than 9 kV, no more than 8 kV, no more than 7 kV, no more than 6 kV, no more than 5 kV, no more than 4 kV, no more than 3 kV, no more than 2 kV, and/or no more than 1 kV.

Shock wave energy generator 930 may be configured to deliver the voltage pulses at a rate of between 1 Hz and 100 Hz, including 1 Hz and 100 Hz, and/or between 1 Hz and 50 Hz, including 1 Hz and 50 Hz. Shock wave energy generator 930 may be configured to deliver the voltage pulses at a rate of a rate of up to 100 Hz, up to 90 Hz, up to 80 Hz, up to 70 Hz, up to 60 Hz, up to 50 Hz, up to 40 Hz, up to 30 Hz, up to 20 Hz, and/or up to 10 Hz. Shock wave energy generator 830 may be configured to deliver the voltage pulses at a rate of at least 10 Hz, at least 20 Hz, at least 30 Hz, at least 40 Hz, at least 50 Hz, at least 60 Hz, at least 70 Hz, at least 80 Hz, at least 90 Hz, and/or at least 100 Hz. Shock wave energy generator 930 may be configured to apply an alternating current to the electrodes of the shock wave emitters to induce a change in the polarity of the electrodes.

Catheter 920 may be connected to fluid source 940. For instance, the catheter may be connected to fluid source 940 via a pump 950, or may be connected to a pressurized fluid sources such as a saline bag. The fluid source 940 may contain a conductive fluid such as saline and/or contrast agent that can be injected into catheter 920, for instance using the pump 950. As described above, the conductive fluid injected into catheter 920 may be used to fill an inflatable enclosure, such as a cap or an angioplasty balloon. The conductive fluid may aid in cooling the device and dissipating heat generated during the formation of vapor bubbles that result from shock wave generation. The enclosure may also shield the shock wave emitters from direct contact with vessel walls and/or direct contact with an occlusion or calcification inside a body lumen.

The catheters described herein may be used for treating various calcifications within a human body lumen, according to some embodiments. For instance, the shock waves generated by any of catheters 100, 200, 300, 400, 500, 600, 700, and/or 920 may be used to crack, fragment, or otherwise break up calcifications within a blood vessel. FIG. 10 illustrates an exemplary method for emitting shock waves in a body lumen, according to some embodiments.

At block 1002, an exemplary catheter is positioned in a body lumen adjacent to a target treatment area. The catheter may include an emitter band that forms at least three shock wave emitters, for instance as described throughout. The catheter may be any of catheters 100, 200, 300, 400, 500, 600, 700 and/or 920 described above. The target treatment area may include circumferential calcium, particularly calcifications of eccentric and nodular morphologies, or other obstructions or concretions. The catheter may be positioned within the vessel such that one or more shock wave emitters face the treatment area. In some examples, prior to positioning of the shock wave emitters such that they face the target treatment area, an imager (e.g., camera) of the catheter may be used to image the lesion for eccentricity or other morphological characteristics. A user (e.g., physician) may position the catheter based the morphology of the lesion and/or select which emitters and/or side of catheter to utilize to generate one or more shock waves in order to optimize treatment based on the morphology of the lesion. Thus, physicians the exemplary catheters described herein may mitigate the need to design a catheter of high torque strength and may also mitigate the need for additional physician training to position the emitters because the catheters described herein may be configured to generate shock waves about the full circumference of the catheter body. For instance, if shock wave emitters were only positioned on one side of an emitter band, a higher sonic output may be generated on one side of the band, but not the other. Thus, such a design may require a physician to learn to “aim” the high energies at the hard to crack calcium. Using the catheters described herein, such “aiming” of the high energies may not be required because they can be generated in a plurality of directions around the catheter.

At block 1004, at least one but not all of the at least three shock wave emitters are pulsed/driven so that shock waves are emitted from the at least one shock wave emitter and not from at least one other of the shock wave emitters. For instance, as described throughout at least one shock wave will be emitted from both a shock wave emitter connected to a supply channel of the voltage source, and at least one other shock wave will be emitted from a shock wave emitter connected to a ground terminal. However. one or more shock wave emitters connected to different supply channels (e.g., those that are not being pulsed/driven) will not emit shock waves. As described above, the shock waves are generated by applying a voltage differential across electrode pairs. A spark is generated as a current flows between the electrodes of the electrode pair across the spark gap, which generates a shock wave. This shock wave generation process may occur simultaneously across a plurality of shock wave emitters connected in series and/or shock waves may be generated at one or more shock wave emitters configured to generate shock waves independently of one or more of the other emitters.

At block 1006, the catheter is optionally advanced further into the blood vessel, for instance, if the first plurality of shock waves did not successfully break up the entire calcification, blockage, fibrotic tissue region, etc. At block 1008, at least one but not all of the at least three shock wave emitters are pulsed/driven again so that shock waves are emitted from the at least one shock wave emitter and not from at least one other of the shock wave emitters. Blocks 1006 and 1008 may be iteratively repeated until the occlusion has been successfully treated.

It should be understood that the aforementioned steps do not need be performed in the order presented, and some steps may be omitted altogether. For instance, the block 1008 may be performed immediately following the initial shock waves generated at step 1004 without advancing the catheter further into the vessel.

As described throughout, shock wave emitters may be positioned at various circumferential locations of a catheter corresponding to apertures formed in an emitter band. For instance, a single emitter band may include at least 3 apertures and/or at least 4 apertures corresponding to at least 3 shock wave emitters and/or at least 4 shock wave emitters. The apertures may be positioned at the same longitudinal location, or different longitudinal locations and spaced about a longitudinal axis of the emitter band. For example, the apertures may be equally spaced by 120 degrees when an emitter band includes three apertures, and equally spaced by 90 degrees when an emitter band includes 4 apertures. The ability to readily generate shock waves using up to four spaced-apart shock wave emitters on a single emitter band may enable more effective treatment of eccentric and nodular calcifications within the vasculature.

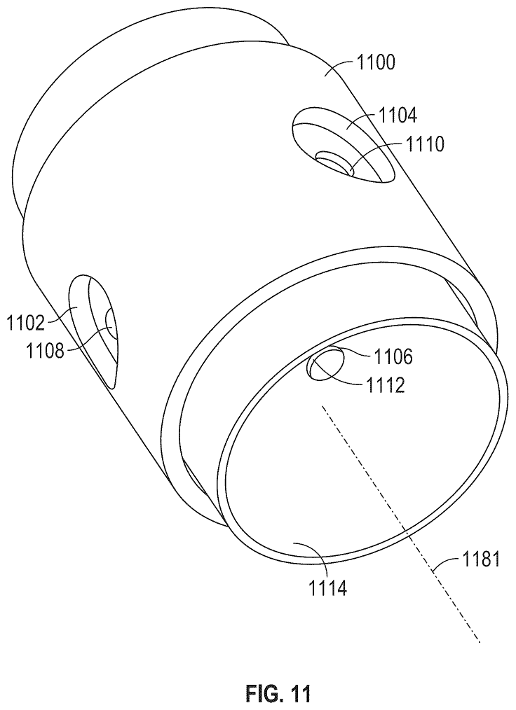

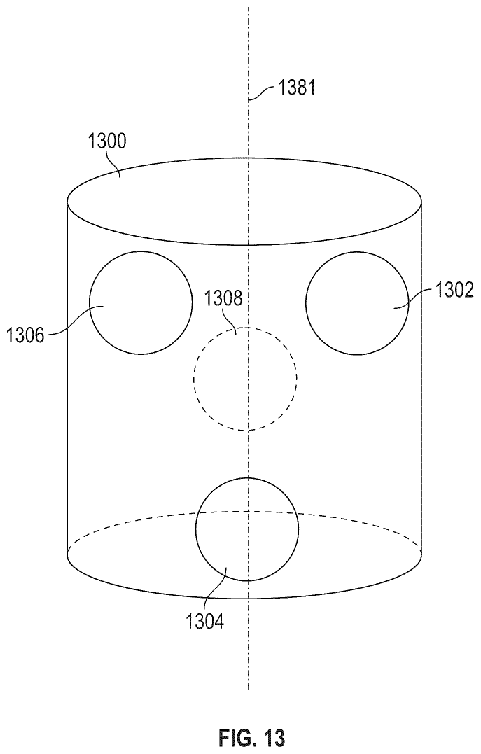

FIG. 11 illustrates an emitter band 1100 that includes three apertures 1102, 1104, and 1106. The apertures are spaced 120 degrees from one another about a longitudinal axis 1181 of the emitter band 1100. However, it should be understood that the apertures could be positioned at any respective positions on the emitter band. For instance, two of the apertures may be spaced 90 degrees apart from one another and each may respectively be spaced apart from a third aperture by 270 degrees. It should be understood that any spacing configuration is within the scope of this disclosure. Additionally, while FIG. 11 depicts each of the apertures at the same longitudinal location, the apertures may instead be positioned at different longitudinal locations of the emitter band 1100, for instance, as shown in FIG. 13.

Emitter band 1100 may be a conductive cylindrical band that surrounds at least a portion of a catheter body (e.g., of any of the catheters described herein.) An insulating sleeve 1114 may be positioned on the inner side of the emitter band 1100 (i.e., between the emitter band 1100 and any of the catheters described herein). The insulating sleeve may ensure that the electrodes of the respective shock wave emitters of the catheters described herein remain electrically isolated from one another. However, the insulating sleeve includes apertures 1108, 1110, and 1112 that respectively correspond to the positions of the apertures 1102, 1104, and 1106 of the emitter band 1100. Accordingly, a first electrode may be positioned at least partially within (e.g., aligned with the opening of) both aperture 1102 of emitter band 1100 and 1108 of insulating sleeve 1114, a second electrode may be positioned at least partially within both aperture 1104 of emitter band 1100 and 1110 of insulating sleeve 1114, and a third electrode may be positioned at least partially within both aperture 1106 of emitter band 1100 and 1112 of insulating sleeve 1114. Thus, a spark gap can be maintained between each of the respective electrodes and the emitter band 1100 while ensuring that electrodes remain electrically isolated from one another.

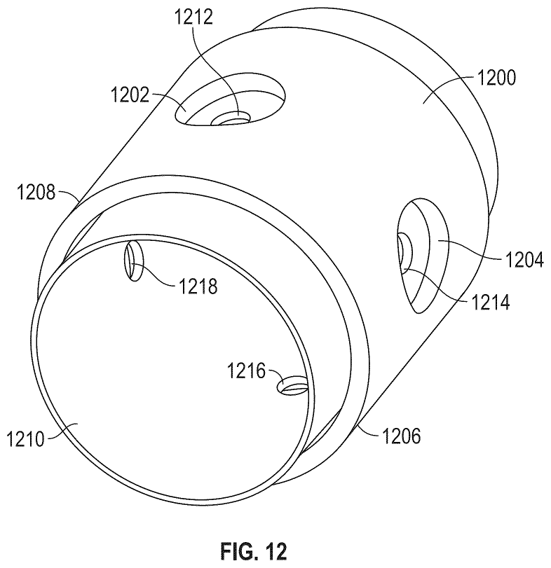

FIG. 12 illustrates an emitter band 1200, similar to emitter band 1200 but having four equally spaced apertures 1202, 1204, 1206, and 1208. As shown, apertures 1202, 1204, 1206, and 1208 are spaced apart from one another by 90 degrees. However, as discussed with respect to the three aperture configuration depicted in FIG. 11, it should be understood that the apertures could be positioned at any respective positions on the emitter band. Emitter band 1200, like emitter band 1100, may be a conductive cylindrical band that surrounds at least a portion of a catheter body (e.g., of any of the catheters described herein.) An insulating sleeve 1210 may be positioned on the inner side of the emitter band 1200 (e.g., between the emitter band 1200 and any of the catheters described herein). The insulating sleeve may ensure that the electrodes of the respective shock wave emitters of the catheters described herein remain electrically isolated from one another. However, the insulating sleeve includes apertures 1212, 1214, 1216, and 1218 that respectively correspond to the positions of the apertures 1202, 1204, 1206, and 1208 of the emitter band 1200. Accordingly, a first electrode may be positioned at least partially within both aperture 1202 of emitter band 1200 and 1212 of insulating sleeve 1210. A second electrode may be positioned at least partially within both aperture 1204 of emitter band 1200 and 1214 of insulating sleeve 1210. A third electrode may be positioned at least partially within both aperture 1206 of emitter band 1200 and 1216 of insulating sleeve 1210. Finally, a fourth electrode may be positioned at least partially within both aperture 1208 of emitter band 1200 and 1218 of insulating sleeve 1210. Thus, a spark gap can be maintained between each of the respective electrodes and the emitter band 1200 while ensuring that electrodes remain electrically isolated from one another.

FIG. 13 illustrates an exemplary emitter band 1300 including a plurality of apertures spaced irregularly about the emitter band. The irregular arrangement may be desirable for treating particularly eccentric, nodular, or otherwise non-uniformly distributed calcium deposits in the vasculature. The apertures may also be arranged to control for emitter degradation and thus lifespan extension. As shown, emitter band 1300 includes four apertures 1302, 1304, 1306, and 1308 positioned at different locations on the emitter band. Apertures 1302 and 1306 are spaced apart from one another about longitudinal axis 1381 (e.g., about the circumference of emitter band 1300) and positioned at the same longitudinal location of emitter band 1300. Aperture 1308 is spaced apart from both apertures 1302 and 1306 about the longitudinal axis 1381 and positioned at a different longitudinal location from both apertures 1302 and 1306. Finally, aperture 1304 is spaced apart from each of apertures 1302, 1306, and 1308 about the longitudinal axis 1381. Aperture 1304 is spaced apart from aperture 1308 by approximately 180 degrees, and is positioned at a different longitudinal location from each of apertures 1302, 1306, and 1308.

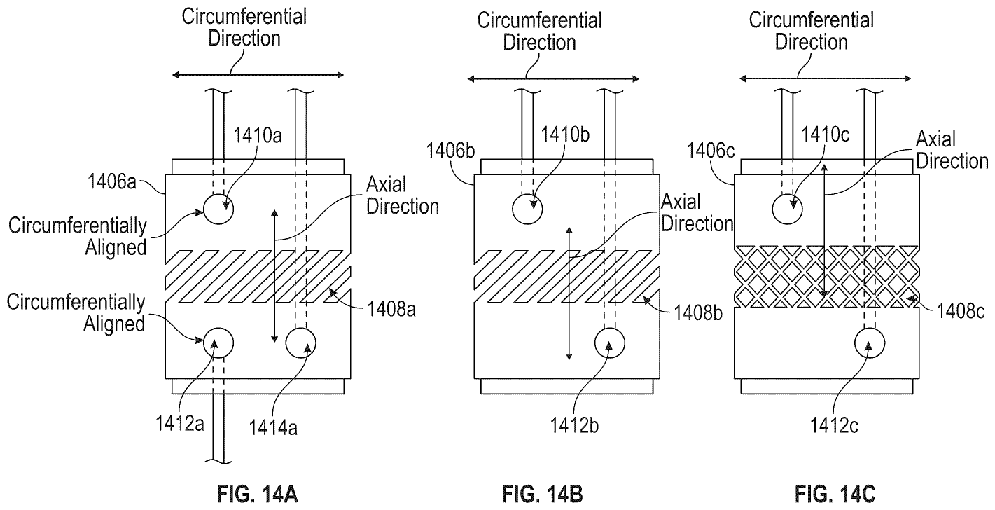

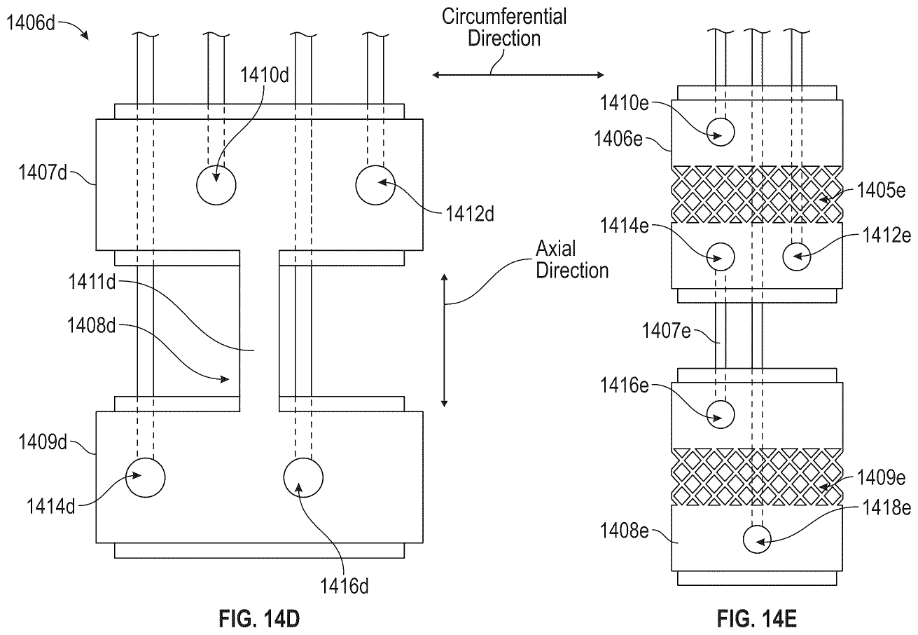

FIGS. 14A-14C depict emitter bands 1406a-1406c. Emitter bands 1406a-1406c include a plurality of openings 1408a-1408c, according to aspects of the disclosure. The plurality of openings may be formed as a plurality of slits, that may extend diagonally (e.g., slits 1408a and 1408b, as shown in FIGS. 14A and 14B), longitudinally, or circumferentially. The plurality of openings 1408a-1408c may extend through the entire thickness of the emitter bands or may comprise a plurality of thinned regions of the emitter bands. Advantageously, the plurality of openings may help to improve the flexibility of relatively longer emitter bands (e.g., emitter bands having lengths greater than or equal to 3 millimeters (mm)) and make the catheter more navigable through tortuous body lumens. The plurality of openings may be formed by laser cutting, etching, or another method.

The emitter bands 1406a-1406c may each form an electrode of a plurality of shock wave emitters. The plurality of shock wave emitters formed in part by each of the emitter bands 1406a-1406c may be axially and/or circumferentially spaced apart from one another on a respective emitter band. Emitter band 1406a, depicted in FIG. 14A, forms an electrode of three shock wave emitters, 1410a, 1412a, and 1414a. Shock wave emitters 1410a and 1412a are axially spaced apart but circumferentially aligned on emitter band 1406a. Shock wave emitter 1414a is circumferentially offset from both shock wave emitters 1410a and 1412a but is axially aligned with shock wave emitter 1412a. Emitter bands 1406b and 1406c depicted in FIGS. 14B and 14C each form an electrode of two shock wave emitters, 1410b and 1412b, and 1410c and 1412c, respectively. Shock wave emitters 1410b and 1412b are axially and circumferentially spaced apart from one another on the emitter back 1406b. Shock wave emitters 1410c and 1412c are axially and circumferentially spaced apart from one another on the emitter back 1406c.

An emitter band having axially spaced shock wave emitters, such as those shown in FIGS. 13 and 14A-14C, may be advantageous over a plurality of axially spaced apart emitter bands without axially spaced shock wave emitters. For example, during manufacturing, assembly of a catheter with a single emitter band (e.g., with a pair of axially spaced apart shock wave emitters) may be more efficient and reproducible than assembly of a catheter with a pair of emitter bands (and electrically connecting elements) with the same number of spaced apart shock wave emitters. Moreover, because the axially spaced shock wave emitters are electrically connected by the emitter band, additional wiring to connect the two emitters is not needed (as would be required if the axially spaced emitters were formed using two separate emitter bands.

It may also be possible to more closely position axially spaced shock wave emitters in a single emitter band and better control constructive interference of shock waves than with a plurality of emitter bands. Shock wave emitters, in some embodiments, may be axially offset (offset in a longitudinal direction of the catheter) by 5 millimeters (mm) or less from each other and configured to generate constructively interfering shock waves. In some embodiments, shock wave emitters may be axially offset by 3 millimeters (mm) or less from each other and configured to generate constructively interfering shock waves. Shock wave emitters may be axially offset by at least one millimeter, at least two millimeters, at least three millimeters, at least four millimeters, and/or at least five millimeters. Shock wave emitters may be axially offset by at most one millimeter, at most two millimeters, at most three millimeters, at most four millimeters, and/or at most five millimeters. Shock wave emitters may be circumferentially aligned or circumferentially offset. Additionally, a single longer emitter band may be desirable or a plurality of electrically connected emitter bands when high voltages (e.g., 10 kV or greater) are applied leading to faster degradation of electrically conducting materials (e.g., joints and wires).

FIG. 14D depicts another emitter band 1406d including a proximal band portion 1407d, a distal band portion 1409d, and a connecting region 1408d. The connecting region may include one or more bridge elements 1411d that may be integrally formed with the proximal and distal band portions. Similar to the plurality of openings in the emitter bands of FIGS. 14A-14C, the connecting region may improve flexibility of the emitter band and thus navigability of the catheter. The connecting region 1408d may include a relatively smaller amount of material than the proximal band portion 1407d and a distal band portion 1409d. For instance, as illustrated in the top-down view of emitter band 1406, the bridge element 1411d may be a straight bracket. The proximal band portion may be cylindrical forming a cylindrical proximal band portion. The distal band portion may be cylindrical forming a cylindrical distal band portion. The bracket may connect the cylindrical proximal band portion to the cylindrical distal band portion. In some examples, connecting region 1408d includes bridge elements 1411d spaced apart from one another 90 degrees about the circumference of the emitter band 1406d. In some examples, connecting region 1408d includes a surface area of less than fifty percent (50%) of the proximal band portion 1407d and/or the distal band portion 1409d. In some examples, connecting region 1408d includes a surface area of less than forty percent (40%) of the proximal band portion 1407d and/or the distal band portion 1409d. In some examples, connecting region 1408d includes a surface area of less than thirty percent (30%) of the proximal band portion 1407d and/or the distal band portion 1409d. In some examples, connecting region 1408d includes a surface area of less than twenty percent (20%) of the proximal band portion 1407d and/or the distal band portion 1409d. In some examples, connecting region 1408d includes a surface area of less than ten percent (10%) of the proximal band portion 1407d and/or the distal band portion 1409d.

Like the emitter bands shown in FIGS. 14A-14C, the emitter band of FIG. 14D includes a plurality of shock wave emitters 1410d, 1412d, 1414d, and 1416d. Shock wave emitters 1410d and 1412d are positioned on the proximal band portion 1407d. Shock wave emitters 1410d and 1412d are axially aligned with, and circumferentially offset from, one another. Shock wave emitters 1414d and 1416d are positioned on the distal band portion 1409d. Shock wave emitters 1410d and 1412d are axially spaced apart from shock wave emitters 1414d and 1416d. The shock wave emitters 1410d, 1412d, 1414d, and 1416d may be configured such that shock waves generated by two or more of the emitters 1410d, 1412d, 1414d, and 1416d constructively interfere with one another as they propagate away from the respective emitters.

FIG. 14E illustrates how relatively longer emitter bands, such as those depicted in FIGS. 14A-14D may be connected on a catheter. By including such a high concentration of shock wave emitters at one region of a catheter, it may be possible to more efficiently treat larger vessels or structural heart features. FIG. 14E depicts a pair of emitter bands 1406e and 1408e having a plurality of openings, the pair of emitter bands 1406e and 1408e electrically connect together by a conductive element 1407e (e.g., a wire). Emitter band 1406e may include a plurality of shock wave emitters 1410e, 1412e and 1414e. Shock wave emitter 1410e may be axially spaced apart from, and circumferentially aligned with, shock wave emitter 1414c. Shock wave emitter 1412e may be axially aligned with, and circumferentially offset from shock wave emitter 1414e. Shock wave emitter 1414e may be electrically connected to a shock wave emitter 1416e positioned at emitter band 1408e via conductive element 1407e extending between emitter band 1406c and 1408c. Each of emitter bands 1406c and 1408e may include a plurality of openings 1405e and 1409e, respectively, according to aspects of the disclosure. The plurality of openings may be formed as a plurality of slits, that may extend diagonally (e.g., slits 1408a and 1408b, as shown in FIGS. 14A and 14B), longitudinally, or circumferentially. The plurality of openings may extend through the entire thickness of the emitter bands 1406e and/or 1408e or may comprise a plurality of thinned regions of the emitter bands. The plurality of openings may help to improve the flexibility of relatively longer emitter bands (e.g., emitter bands having lengths greater than 3 millimeters (mm)) and make the catheter more navigable through tortuous body lumens. The plurality of openings may be formed by laser cutting, etching, or another method.

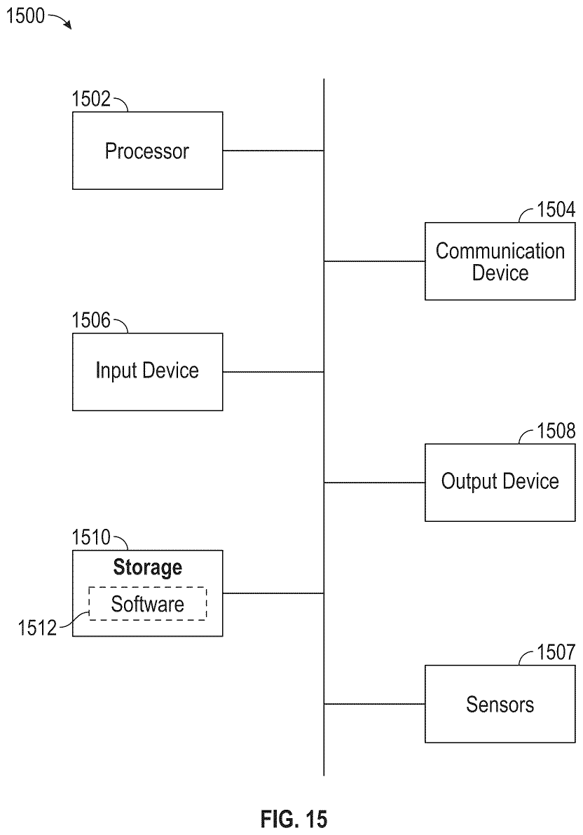

FIG. 15 depicts an exemplary computing device 1500 which may form part of the system 100 described above and may be used for performing various steps of the methods described herein, in accordance with one or more examples of the disclosure. Device 1500 can be a host computer connected to a network. Device 1500 can be a client computer or a server. As shown in FIG. 15, device 1500 can be any suitable type of microprocessor-based device, such as a personal computer, workstation, server, or handheld computing device (i.e., a portable electronic device) such as a phone or tablet. The device can include, for example, one or more processors 1502, input device 1506, sensor device 1507, output device 1508, storage 1510, and communication device 1504. Input device 1506 and output device 1508 can generally correspond to those described above and can either be connectable or integrated with the computer.

Input device 1506 can be any suitable device that provides directed input, such as a touch screen, keyboard or keypad, mouse, or voice-recognition device, in other words, input or directions provided or initiated by a user. Sensor device 1507 can be one or more of any suitable sensor devices, such as a pressure sensor, a thermal sensor, an electrical sensor (e.g., current, voltage, resistance, and/or impedance sensors), or a visualization element. Output device 1508 can be any suitable device that provides output, such as a touch screen, haptics device, or speaker. Storage 1510 can be any suitable device that provides storage, such as an electrical, magnetic, or optical memory, including a RAM, cache, hard drive, or removable storage disk. Communication device 1504 can include any suitable device capable of transmitting and receiving signals over a network, such as a network interface chip or device. The components of the computer can be connected in any suitable manner, such as via a physical bus or wirelessly.

Sensor devices 1507 can provide feedback to an operator using device 1500 by measuring parameters in the surrounding environment and thereby indicating a status of a shock wave catheter device such as catheter 10 connected to computing device 1500, and further providing for guidance on what further steps the operator may decide to implement a shock wave catheter device such as catheter 10 connected to computing device 1500. For example, in implementations where sensor devices 1507 include pressure sensors, a slight decrease in pressure may indicate success at cracking a calcified lesion, due to the fact that an expandable member (e.g., enclosure 120) surrounding shock wave emitters is able to further expand without changing the volume of fluid within the expandable member. Further, a significant decrease in pressure may indicate a rupture failure mode where the expandable member has lost seal and fluid volume, and thus guiding toward withdrawal of the device (e.g., device 10). In implementations where the sensor devices include a visualization element, an operator of a catheter device such as catheter 10 may be able to more clearly understand where the catheter is located relative to a target lesion or anatomy, prior to, during, and after delivering therapy.

In some embodiments, sensor device 1507 includes surface electrodes of an electrocardiograph to synchronize a shock wave to the “R” wave for treating vessels near the heart. Sensor device 1507 may include an R-wave detector and a controller to control the high voltage switch. Mechanical shocks can stimulate heart muscle and could lead to an arrhythmia. While it is unlikely that shock waves of such short duration as contemplated herein would stimulate the heart by synchronizing the pulses (or bursts of pulses) with the R-wave, an additional degree of safety may be provided when used on vessels of the heart or near the heart. In implementations where shock waves are generated from open unenclosed emitters, synchronization to the R-wave would significantly improve the safety against unwanted arrhythmias.

Software 1512, which can be stored in storage 1510 and executed by processor 1502, can include, for example, the programming that embodies the functionality of the present disclosure (e.g., as embodied in the devices as described above). Software 1512 can also be stored and/or transported within any non-transitory computer-readable storage medium for use by, or in connection with, an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1510, that can contain or store programming for use by, or in connection with, an instruction execution system, apparatus, or device. Software 1512 can also be propagated within any transport medium for use by, or in connection with, an instruction execution system, apparatus, or device, such as those described above, that can fetch instructions associated with the software from the instruction execution system, apparatus, or device and execute the instructions. In the context of this disclosure, a transport medium can be any medium that can communicate, propagate, or transport programming for use by, or in connection with, an instruction execution system, apparatus, or device. The transport-readable medium can include, but is not limited to, an electronic, magnetic, optical, electromagnetic, or infrared wired or wireless propagation medium.

Device 1500 may be connected to a network, which can be any suitable type of interconnected communication system. The network can implement any suitable communication protocols and can be secured by any suitable security protocols. The network can comprise network links of any suitable arrangement that can implement the transmission and reception of network signals, such as wireless network connections, T1 or T3 lines, cable networks, DSL, or telephone lines. Device 1500 can implement any operating system suitable for operating on the network. Software 1512 can be written in any suitable programming language, such as C, C++, Java, or Python. In various embodiments, application software embodying the functionality of the present disclosure can be deployed in different configurations, such as in a client/server arrangement or through a Web browser as a Web-based application or Web service, for example.

Although the electrode assemblies and catheter devices described herein have been discussed primarily in the context of treating coronary occlusions, such as lesions in vasculature, the electrode assemblies and catheters herein can be used for a variety of occlusions, such as occlusions in the peripheral vasculature (e.g., above-the-knee, below-the-knee, iliac, carotid, etc.). For further examples, various embodiments may be used for treating soft tissues, such as cancer and tumors (i.e., non-thermal ablation methods), blood clots, fibroids, cysts, organs, scar and fibrotic tissue removal, or other tissue destruction and removal. Electrode assembly and catheter designs could also be used for neurostimulation treatments, targeted drug delivery, treatments of tumors in body lumens (e.g., tumors in blood vessels, the esophagus, intestines, stomach, or vagina), wound treatment, non-surgical removal and destruction of tissue, or used in place of thermal treatments or cauterization for venous insufficiency and fallopian ligation (i.e., for permanent female contraception).

In one or more examples, the electrode assemblies and catheters described herein could also be used for tissue engineering methods, for instance, for mechanical tissue decellularization to create a bioactive scaffold in which new cells (e.g., exogenous or endogenous cells) can replace the old cells; introducing porosity to a site to improve cellular retention, cellular infiltration/migration, and diffusion of nutrients and signaling molecules to promote angiogenesis, cellular proliferation, and tissue regeneration similar to cell replacement therapy. Such tissue engineering methods may be useful for treating ischemic heart disease, fibrotic liver, fibrotic bowel, and traumatic spinal cord injury (SCI). For instance, for the treatment of spinal cord injury, the devices and assemblies described herein could facilitate the removal of scarred spinal cord tissue, which acts like a barrier for neuronal reconnection, before the injection of an anti-inflammatory hydrogel loaded with lentivirus to genetically engineer the spinal cord neurons to regenerate.

It should be noted that the elements and features of the example catheters illustrated throughout this specification and drawings may be rearranged, recombined, and modified without departing from the present invention. For instance, while this specification and drawings describe and illustrate catheters having several example balloon designs, the present disclosure is intended to include catheters having a variety of balloon configurations. The number, placement, and spacing of the electrode pairs of the shock wave generators can be modified without departing from the subject invention. Further, the number, placement, and spacing of balloons of catheters can be modified without departing from the subject invention.

It should be understood that the foregoing is only illustrative of the principles of the invention, and that various modifications, alterations and combinations can be made by those skilled in the art without departing from the scope and spirit of the invention. Any of the variations of the various catheters disclosed herein can include features described by any other catheters or combination of catheters herein. Furthermore, any of the methods can be used with any of the catheters disclosed. Accordingly, it is not intended that the invention be limited, except as by the appended claims.

1. A catheter for generating shock waves comprising:
a catheter body;
an enclosure mounted to the catheter body;
an emitter band positioned at least partially around the catheter body and within the enclosure; and
at least three electrodes positioned adjacent to the emitter band and spaced apart from the emitter band by respective spark gaps, the at least three electrodes together with the emitter band forming electrode pairs of at least three shock wave emitters of the emitter band, wherein at least one of the at least three shock wave emitters can be driven separately from at least one other of the at least three shock wave emitters.

2. The catheter of claim 1, wherein a first shock wave emitter is electrically connected in series to a second shock wave emitter of the at least three shock wave emitters, a third shock wave emitter is electrically connected in series to the second shock wave emitter, and the first shock wave emitter and the third shock wave emitter are not electrically connected in series.

3. The catheter of claim 2, wherein a fourth shock wave emitter of the at least three shock wave emitters is electrically connected in series to the second shock wave emitter.

4. The catheter of claim 1, wherein the emitter band comprises at least three apertures corresponding to the at least three electrodes.

5. The catheter of claim 4, wherein the at least three apertures are respectively spaced apart 120-degrees from one another in a circumferential direction about the emitter band.

6. The catheter of claim 4, wherein the emitter band comprises four apertures spaced apart 90 degrees from one another in a circumferential direction about the emitter band.

7. The catheter of claim 6, wherein the four apertures correspond to four shock wave emitters.

8. The catheter of claim 1, wherein a first electrode is connected to a first channel of a switching circuit by a first supply wire extending along the catheter body, and a second electrode is connected to a second channel of a switching circuit by a second supply wire extending along the catheter body.

9. The catheter of claim 1, wherein at least two of the at least three shock wave emitters are spaced apart from one another in a circumferential direction about the emitter band by any of 60 degrees, 72 degrees, 90 degrees, or 120 degrees.

10. The catheter of claim 1, wherein at least two of the at least three shock wave emitters are spaced apart from one another in a circumferential direction about the emitter band by a range of 60 degrees to less than 180 degrees.

11. The catheter of claim 1, comprising a second emitter band that forms at least one electrode of at least one shock wave emitter of the second emitter band, wherein at least one of the at least three electrodes is electrically connected to an electrode of the at least one shock wave emitter of the second emitter band.

12. The catheter of claim 11, wherein the second emitter band is positioned distally of the emitter band.

13. The catheter of claim 11, wherein at least one of the at least one shock wave emitter of the second emitter band is configured to be driven independently of at least one other shock wave emitter of the second emitter band.

14. The catheter of claim 1, wherein at least one of the at least three shock wave emitters is positioned distally of at least one other of the at least three shock wave emitters.

15. The catheter of claim 1, wherein the enclosure is a balloon configured to be filled or inflated with a conductive fluid.

16. The catheter of claim 1, wherein the at least three electrodes respectively comprise a conductive portion of at least three insulated wires.

17. The catheter of claim 1, wherein the emitter band is at least 3 millimeters long.

18. The catheter of claim 1, wherein a first shock wave emitter is axially offset from a second shock wave emitter by 1 millimeter to 5 millimeters.

19. The catheter of claim 1, wherein a first shock wave emitter is axially offset from a second shock wave emitter by 1 millimeter to 3 millimeters.

20. The catheter of claim 1, wherein the emitter band comprises a proximal band portion, a distal band portion, and a connecting region between the proximal band portion and the distal band portion.

21. The catheter of claim 20, wherein the connecting region comprises a bracket that connects the proximal band portion and the distal band portion, wherein the proximal band portion forms a cylindrical proximal band portion and the distal band portion forms a cylindrical distal band portion.

22. The catheter of claim 1, wherein the emitter band comprises a plurality of openings positioned between a first shock wave emitter of the at least three shock wave emitters and a second shock wave emitter of the at least shock wave emitters.

23. The catheter of claim 22, wherein the plurality of openings are positioned distally of the first shock wave emitter and proximally of the second shock wave emitter.

24. The catheter of claim 22, wherein the plurality of openings are formed using a laser cutter.

25. The catheter of claim 1, wherein the emitter band comprises a plurality of thinned regions positioned between a first shock wave emitter of the at least three shock wave emitters and a second shock wave emitter of the at least shock wave emitters.

26. The catheter of claim 25, wherein the plurality of thinned regions are positioned distally of the first shock wave emitter and proximally of the second shock wave emitter.

27. A system for generating shock waves comprising:
the catheter of claim 1; and
a shock wave energy generator for generating energy pulses for driving the at least three shock wave emitters, the pulse generator comprising a switching circuit for driving at least one of the shock wave emitters independently of at least one other shock wave emitter of the at least three shock wave emitters.

28. The system of claim 27, wherein the at least one shock wave emitter and the at least one other shock wave emitter are respectively connected to a first channel and a second channel of the switching circuit by a first supply wire and a second supply wire.

29. The system of claim 27, wherein at least one of the at least three shock wave emitters is connected to a ground terminal of the switching circuit.

30. The system of claim 27, wherein the shock wave energy generator is configured to deliver high voltage pulses to at least one of the at least three shock wave emitters.

31. A method for treating a target area in a body lumen comprising:
positioning a catheter in the body lumen adjacent to the target area, the catheter comprising an emitter band that forms at least three shock wave emitters; and
driving at least one but not all of the at least three shock wave emitters so that shock waves are emitted from the at least one shock wave emitter and not from at least one other of the at least three shock wave emitters.

32. The method of claim 31, wherein driving the at least one shock wave emitter causes at least two shock wave emitters of the emitter band to generate a respective shock wave.

33. The method of claim 32, wherein the at least two shock wave emitters that generate a respective shock wave comprise a shock wave emitter connected to a negative terminal of a voltage source and a different shock wave emitter connected to ground.

34. The method of claim 31, wherein driving the at least one shock wave emitter causes at least one other shock wave emitter of a second emitter band to generate a shock wave.

35. A catheter comprising:
a catheter body,
an enclosure mounted to the catheter body;
an emitter band positioned at least partially around the catheter body and within the enclosure;
a first shock wave emitter connected to a first channel of a power supply, the first shock wave emitter comprising at least one electrode pair, the at least one electrode pair comprising the emitter band; and
a second shock wave emitter connected to a second channel of the power supply so that the second shock wave emitter can be driven independently of the first shock wave emitter, the second shock wave emitter comprising at least one electrode pair that comprises the emitter band.

36. The catheter of claim 35, comprising a third shock wave emitter connected to a ground terminal, the third shock wave emitter comprising at least one electrode pair, the at least one electrode pair comprising the emitter band.

37. The catheter of claim 35, comprising a second emitter band electrically connected to the emitter band.
